SEQUENCE LISTING (42 sequences)

SEQ ID NO: 1 - DNA, 33 bp, Artificial Sequence
  primer
taaacattcatattatttcaaaaaatttgaaac

SEQ ID NO: 2 - DNA, 24 bp, Artificial Sequence
  primer
tgccctcagaacttgcctcagcgc

SEQ ID NO: 3 - DNA, 48 bp, Artificial Sequence
  primer
attcgtatccaagattaaacattcatattatttcaaaaaatttgaaac

SEQ ID NO: 4 - DNA, 32 bp, Artificial Sequence
  primer
tcatcgacgcttaagaaaaatgcataaatgct

SEQ ID NO: 5 - DNA, 40 bp, Artificial Sequence
  primer
gaattcgcagctactaggagtttgtaaatgaagtacagtt

SEQ ID NO: 6 - DNA, 40 bp, Artificial Sequence
  primer
tagtgtatccaagtatgccctcagaacttgcctcagcgca

SEQ ID NO: 7 - DNA, 24 bp, Artificial Sequence
  primer
caccgcatttttcttaagcgtcga

SEQ ID NO: 8 - DNA, 24 bp, Artificial Sequence
  primer
aaactcgacgcttaagaaaaatgc

SEQ ID NO: 9 - DNA, 62 bp, Artificial Sequence
  primer
ggggacaagtttgtacaaaaaagcaggcttaaacattcatattatttcaaaaaatttgaaac

SEQ ID NO: 10 - DNA, 54 bp, Artificial Sequence
  primer
ggggaccactttgtacaagaaagctgggttgccctcagaacttgcctcagcgca

SEQ ID NO: 11 - DNA, 40 bp, Artificial Sequence
  primer
tggaattctgcagatgccaccatgactgaatataaacttg

SEQ ID NO: 12 - DNA, 35 bp, Artificial Sequence
  primer
gccactgtgctggatttacattataatgcattttt

SEQ ID NO: 13 - DNA, 21 bp, Artificial Sequence
  sh-KRAS target sites
atggtcctagtaggaaataaa

SEQ ID NO: 14 - DNA, 21 bp, Artificial Sequence
  sh-KRAS-target sites
ttgatgatgccttctatacat

SEQ ID NO: 15 - DNA, 20 bp, Artificial Sequence
  primer
agtacccagggacctccatc

SEQ ID NO: 16 - DNA, 20 bp, Artificial Sequence
  primer
gtgtgcagggtcagtctctc

SEQ ID NO: 17 - DNA, 20 bp, Artificial Sequence
  primer
cccctcattttgtttgcggt

SEQ ID NO: 18 - DNA, 20 bp, Artificial Sequence
  primer
tttctcccaaggttctcggc

SEQ ID NO: 19 - DNA, 20 bp, Artificial Sequence
  primer
aatctctacccgcctccctt

SEQ ID NO: 20 - DNA, 20 bp, Artificial Sequence
  primer
cacagagctcgtgaaccagt

SEQ ID NO: 21 - DNA, 19 bp, Artificial Sequence
  primer
cccactccaccatcgtcag

SEQ ID NO: 22 - DNA, 20 bp, Artificial Sequence
  primer
atcctgccatacctgtctgc

SEQ ID NO: 23 - DNA, 20 bp, Artificial Sequence
  primer
ggagtgatgggtctgtggtc

SEQ ID NO: 24 - DNA, 20 bp, Artificial Sequence
  primer
actccgaggcacgttgttta

SEQ ID NO: 25 - DNA, 20 bp, Artificial Sequence
  primer
tgagatcgggagcttcctca

SEQ ID NO: 26 - DNA, 20 bp, Artificial Sequence
  primer
gagccccaagggaatcatgt

SEQ ID NO: 27 - DNA, 21 bp, Artificial Sequence
  primer
ttgccttccaagtgcaggagt

SEQ ID NO: 28 - DNA, 20 bp, Artificial Sequence
  primer
ctctctctgcaccactgcgg

SEQ ID NO: 29 - DNA, 20 bp, Artificial Sequence
  primer
cccaactcaatctgcacagc

SEQ ID NO: 30 - DNA, 20 bp, Artificial Sequence
  primer
ctgaatgacctggcgtggaa

SEQ ID NO: 31 - DNA, 20 bp, Artificial Sequence
  primer
gagctaccacgcagacatca

SEQ ID NO: 32 - DNA, 20 bp, Artificial Sequence
  primer
cgaggaagtagaagcggttg

SEQ ID NO: 33 - DNA, 20 bp, Artificial Sequence
  primer
caagcgggacgagtggctgg

SEQ ID NO: 34 - DNA, 20 bp, Artificial Sequence
  primer
tcagtgggctgctgcacagc

SEQ ID NO: 35 - DNA, 20 bp, Artificial Sequence
  primer
ctcagaggcagtgacagcag

SEQ ID NO: 36 - DNA, 20 bp, Artificial Sequence
  primer
tgtctgaattcccgttctcc

SEQ ID NO: 37 - DNA, 21 bp, Artificial Sequence
  primer
accccctgtcacctgctgagg

SEQ ID NO: 38 - DNA, 20 bp, Artificial Sequence
  primer
gcgatggccaccccaccaat

SEQ ID NO: 39 - DNA, 20 bp, Artificial Sequence
  primer
tcacaccaggtgcacttctc

SEQ ID NO: 40 - DNA, 20 bp, Artificial Sequence
  primer
ggatggttgtgtttgtgctg

SEQ ID NO: 41 - DNA, 20 bp, Artificial Sequence
  primer
tctccccagtcttacctccg

SEQ ID NO: 42 - DNA, 20 bp, Artificial Sequence
  primer
ccaggcttcagggaactcag

(19) United States
(12) Patent Application Publication
(10) Pub. No.: US 2022/0125877 A1
(43) Pub. Date: Apr. 28, 2022

(54) METHOD FOR TREATING COLORECTAL CANCER

(71) Applicant: SUN YAT-SEN UNIVERSITY, Guangdong (CN)

(72) Inventors: Guoquan GAO, Guangdong (CN); Ti ZHOU, Guangdong (CN); Xia YANG, Guangdong (CN); Jinye XIE, Guangdong (CN)

(73) Assignee: SUN YAT-SEN UNIVERSITY, Guangdong (CN)

(21) Appl. No.: 17/571,550

(22) Filed: Jan. 10, 2022

Related U.S. Application Data
(63) Continuation-in-part of application No. PCT/CN2020/099975, filed on Jul. 2, 2020.
Application No. PCT/CN2020/099975 is a continuation-in-part of application No. PCT/CN2020/099974, filed on Jul. 2, 2020.

(30) Foreign Application Priority Data
Jul. 10, 2019 (CN) 201910620014.6
Jul. 10, 2019 (CN) 201910620051.7

(51) Int. Cl. A61K 38/17; A61K 38/45; A61K 31/155; A61P 35/00

(52) U.S. Cl. CPC A61K 38/177; A61P 35/00; A61K 31/155; A61K 38/45

(57) ABSTRACT

The present invention relates to a method for treating colorectal cancer (CRC); this present invention confirms that KRAS mutation CRC patients benefit from metformin more significantly for the first time and further confirms that the down-regulated expression of MATE1 in the discharge passage of metformin is a key mechanism for the sensitivity of KRAS mutation CRC cells to metformin. The transcriptional level of MATE1 is decreased and the concentration of metformin in tumor cells is increased by upregulating the transmethylase DNMT1 and down-regulating the demethylase TET1/2, and thereby the inhibiting effect of metformin on tumor cell proliferation is enhanced. The present invention provides a novel thinking and method for the treatment of colorectal cancer, which has profound significance and is worth popularizing energetically.

### CROSS-REFERENCE TO RELATED APPLICATION

This application is a continuation-in-part of international application of PCT application serial no. PCT/CN2020/099975 filed on Jul. 2, 2020 and PCT application serial no. PCT/CN2020/099974 filed on Jul. 2, 2020 which claim the priority benefit of China application no. 201910620014.6 filed on Jul. 10, 2019 and China application no. 201910620051.7 filed on Jul. 10, 2019. The entirety of each of the above mentioned patent applications is hereby incorporated by reference herein and made a part of this specification.

### BACKGROUND

### Technical Field

The present invention relates to the technical field of cancer therapy, and in particular to a method for treating colorectal cancer.

### Description of Related Art

Colorectal cancer (CRC) is one of the most common malignant tumors. Currently, the combined use of Oxaliplatin or Irinotecan-oriented chemotherapy with epidermal growth factor receptor (EGFR) monoclonal antibodies can increase more than 2 years mean overall survival (OS) of the colorectal cancer patients. However, in China, about 1/4 patients have occurred tumor metastasis when diagnosis confirmed, and thus a chemotherapeutic efficacy is poor. Moreover, colorectal cancer is a kind of genetically heterogeneous disease. The change (mutation or deletion) of genes, such as APC, KRAS, TP53, BRAF, PIK3CA as well as microsatellite instability (MSI) and chromosomal instability (CIN) and other epigenetic alterations play an important role in the process of cancerization from intestinal polyps, tumor metastasis process and chemotherapeutics resistance. In China, KRAS gene mutation probability of colorectal cancer patients is up to 30%-50%. Lots of clinical studies have indicated that such kind of patients do not benefit from anti-EGFR targeted therapy, which thus causes that the CRC tumor-related mortality shows a rapid rising trend in China.

Therefore, it is crucial for the precise tumor treatment to research and develop a therapeutic regimen and a medicament directed to the individual epigenetic alteration of colorectal cancer patients and to be in transition to multi-targeted drug therapy from the conventional chemotherapeutics indiscriminatingly killing cells.

At present, the therapeutic strategy directed to KRAS mutation CRC includes inhibition to KRAS activation or inhibition to the activation of KRAS downstream proliferation signaling pathways MEK/ERK, but the above therapeutic strategy ends in failure of phase II clinic trials. The failure reason of the former one is that the activation of KRAS cannot be completely inhibited by the Farnesylation transferase inhibitor; and the failure reason of the latter one may be associated with the feedback activation of PI3K/AKT signaling pathway.

Metformin is a kind of first-line drug to the treatment of type 2 diabetes mellitus (T2DM) at present, and can effectively lower and maintain patients' blood glucose level and insulin level, thus improving insulin resistance. In recent years, more retrospective studies have showed that metformin has certain prevention and treatment effects on colorectal cancer; the major mechanism of metformin includes direct effects on tumor cells: inhibiting the activation of MEK-ERK, PI3K-AKT and mTOR signaling pathways, and indirect effects on tumor cells: such as, lowering and maintaining blood glucose and insulin level, inhibiting inflammatory reaction, increasing the ratio of CD8+ T cells, thereby improving the cellular immune functions of tumor, and the like. But meanwhile, partial studies have reported that metformin cannot improve the overall survival and progression free survival of colorectal cancer patients.

The above studies prompt that metformin may have type and individual differences on the therapeutic effect of colorectal cancer. The effective treatment type and individual of metformin for colorectal cancer have been not made clear, and the mechanism has not been clarified, either currently.

### SUMMARY

Objectives of the present invention are to overcome the above shortcomings in the prior art, and to provide a method for treating colorectal cancer.

The first objective of the present invention is to provide use of a MATE1 gene and/or protein as a therapeutic target of colorectal cancer.

The second objective of the present invention is to provide a marker for determining the selective use of metformin in the treatment of colorectal cancer.

The third objective of the present invention is to provide use of a MATE1 gene and/or protein as a marker for determining a therapeutic regimen of colorectal cancer.

The fourth objective of the present invention is to provide a kit for determining a therapeutic regimen of colorectal cancer.

The fifth objective of the present invention is to provide use of a MATE1 protein and/or an inhibitor of an encoding gene SLC47A1 thereof in the synergistic treatment of colorectal cancer with metformin or in the preparation of a medicament for the synergistic treatment of colorectal cancer with metformin.

The sixth objective of the present invention is to provide a pharmaceutical composition for treating colorectal cancer.

The seventh objective of the present invention is to provide a method for treating colorectal cancer.

The eighth objective of the present invention is to provide use of a MATE1 protein and/or an inhibitor of an encoding gene SLC47A1 thereof as a synergist for the treatment of colorectal cancer with metformin.

The ninth objective of the present invention is to provide a marker for determining a therapeutic regimen of colorectal cancer.

The tenth objective of the present invention is to provide use of a KRAS gene and/or protein as a marker for determining a therapeutic regimen of colorectal cancer.

The eleventh objective of the present invention is to provide use of a detection reagent of KRAS-gene mutation in the preparation of a kit for determining a therapeutic regimen of colorectal cancer.

The twelfth objective of the present invention is to provide use of a detection reagent of KRAS protein in the preparation of a kit for determining a therapeutic regimen of colorectal cancer.

The thirteenth objective of the present invention is to provide a kit for determining a therapeutic regimen of colorectal cancer.

The fourteenth objective of the present invention is to provide use of metformin in the treatment of colorectal cancer or in the preparation of a medicament for the treatment of colorectal cancer.

The fifteenth objective of the present invention is to provide use of a combination of a MEK signaling pathway and an AKT signaling pathway as a therapeutic target of colorectal cancer.

The sixteenth objective of the present invention is to provide use in combination with an inhibitor of the MEK signaling pathway and an inhibitor of the AKT signaling pathway in the treatment of KRAS mutation colorectal cancer.

The seventeenth objective of the present invention is to provide a pharmaceutical composition for treating KRAS mutation colorectal cancer.

To achieve the above objectives, the present invention is implemented by the following technical solutions.

1. A stratified Cox proportional hazard model is used to perform a retrospective study to confirm that KRAS mutation CRC patients benefit from metformin more significantly for the first time. The present invention provides evidence-based medical evidences for the selective use of metformin in the treatment of colorectal cancer clinically.

2. KRAS (G13D) point mutation model and KRAS knock-down model are constructed on a cell experiment to forward and reversely verify that the KRAS mutation colorectal cancer cell is sensitive to the anti-tumor effect of metformin. It is clarified that metformin inhibits the two pathways of ERK/cyclin D1/RB and AKT/mTOR/4E-BP1 at the same time, thus inhibiting the proliferation of KRAS mutation colorectal cancer cells. The present invention provides evidences for the combined use of MEK and AKT inhibitors to enhance the therapeutic effect of KRAS mutation colorectal cancer cells, and meanwhile provides an experimental evidence for metformin as an alternative drug.

3. It is confirmed for the first time that the down-regulated expression of MATE1 in the discharge passage of metformin is a key mechanism for the sensitivity of KRAS mutation CRC cells to metformin. The present invention clarifies that the promoter CpG island methylation of MATE1 is accelerated and the transcriptional level of MATE1 is decreased by upregulating the transmethylase DNMT1 and down-regulating the demethylase TET1/2.

4. Clinical specimens and cell experimental evidences prompt that metformin can be selectively used to treat colorectal cancer by detecting KRAS genotype clinically.

Therefore, the present invention claims to protect the following contents:

Provided is a marker for determining a therapeutic regimen of colorectal cancer, wherein the marker is a KRAS gene and/or protein.

Provided is use of the KRAS gene and/or protein as a marker for determining a therapeutic regimen of colorectal cancer.

Specifically, the KRAS mutation colorectal cancer is selectively treated by metformin; and for the KRAS mutation colorectal cancer described herein, a KRAS gene-related downstream cell proliferative signaling is persistently activated and highly expressed after mutation.

Use of a detection reagent of KRAS gene mutation in the preparation of a kit for determining a therapeutic regimen of colorectal cancer also falls within the protection scope of the present invention.

Use of a detection reagent of KRAS protein in the preparation of a kit for determining a therapeutic regimen of colorectal cancer also falls within the protection scope of the present invention.

Provided is a kit for determining a therapeutic regimen of colorectal cancer, wherein the kit includes a detection reagent of KRAS mutation colorectal cancer.

Provided is use of metformin in the treatment of colorectal cancer or in the preparation of a medicament for the treatment of colorectal cancer, wherein the colorectal cancer is KRAS mutation colorectal cancer.

The type of KRAS mutations is not limited to the KRAS mutation colorectal cancer described above, and common mutations with experimental data support of codon12.13 are available.

MEK and AKT are important signaling pathways to regulate and control cell proliferation in the downstream of KRAS. The single use of MEK inhibitor or AKT inhibitor ends in failure of phase-II clinical trials, while metformin can inhibit MEK and AKT signaling pathways at the same time. Therefore, metformin can effectively inhibit the proliferation of KRAS mutation colorectal cancer cells.

Therefore, the present invention further claims to protect the following contents.

Provided is use of a combination of a MEK signaling pathway and an AKT signaling pathway as a therapeutic target of colorectal cancer.

Provided is use in combination with an inhibitor of the MEK signaling pathway and an inhibitor of the AKT signaling pathway in the treatment of KRAS mutation colorectal cancer.

Provided is a pharmaceutical composition for treating KRAS mutation colorectal cancer, including an inhibitor of the MEK signaling pathway and an inhibitor of the AKT signaling pathway.

Preferably, the treatment of colorectal cancer described above refers to acceleration of G1 lag phase of colorectal cancer cells, inhibition of colonic cancer cell proliferation, inhibition of tumor size increment, inhibition of tumor weight increment, extension of overall survival of patients and/or extension of progress free survival (PFS) of chemotherapy.

More preferably, the extension of progress free survival (PFS) of chemotherapy refers to the extension of the PFS of first-line chemotherapy.

The inhibitors described above are substances capable of randomly reducing the corresponding protein, gene or signaling pathway.

The present patent further claims to protect use of the MATE1 gene and/or protein as a therapeutic target of colorectal cancer.

Provided is a marker for determining the selective use of metformin for the treatment of colorectal cancer, wherein the marker is a MATE1 gene and/or protein.

Preferably, when 100 ng cDNA is subjected to q-PCR to detect the encoding gene SLC47A1 of MATE1, metformin is merely selected to treat colorectal cancer if Ct>30;

if Ct<30, metformin is used in combination with the MATE1 protein and/or an inhibitor of an encoding gene SLC47A1 thereof for the treatment of colorectal cancer.

The lower the expression of MATE1 is, the better the therapeutic effect of metformin on colorectal cancer in single use is.

The use of the MATE1 gene and/or protein as a marker for determining a therapeutic regimen of colorectal cancer also falls within the protection scope of the present invention.

Provided is a kit for determining a therapeutic regimen of colorectal cancer, wherein the kit includes a detection reagent for the expression situation of a MATE1 gene and/or protein.

Provided is use of a MATE1 protein and/or an inhibitor of an encoding gene SLC47A1 thereof in the synergistic treatment of colorectal cancer with metformin or in the preparation of a medicament for the synergistic treatment of colorectal cancer with metformin.

Preferably, the MATE1 protein and/or the inhibitor of an encoding gene SLC47A1 thereof includes: one or a mixture of several of transmethylase DNMT1 or an encoding gene thereof, a transmethylase DNMT3A or an encoding gene thereof, an activating agent of the transmethylase DNMT1 or the encoding gene thereof, an activating agent of the transmethylase DNMT3A or the encoding gene thereof, an inhibitor of a demethylase TET1, an inhibitor of a demethylase TET2, an inhibitor of the encoding gene of the demethylase TET1, and/or an inhibitor of the encoding gene of the demethylase TET2.

Provided is a pharmaceutical composition for treating colorectal cancer, wherein, the pharmaceutical composition contains metformin, and a MATE1 protein and/or an inhibitor of an encoding gene SLC47A1 thereof.

Provided is a method for treating colorectal cancer, wherein, metformin is used in combination with one or more of a MATE1 protein and/or an inhibitor of an encoding gene SLC47A1 thereof.

Provided is use of a MATE1 protein and/or an inhibitor of an encoding gene SLC47A1 thereof as a synergist for the treatment of colorectal cancer with metformin.

Compared with the prior art, the prevent invention has the following beneficial effects.

The present invention confirms that KRAS mutation CRC patients benefit from metformin more significantly for the first time and further confirms that the down-regulated expression of MATE1 in the discharge passage of metformin is a key mechanism for the sensitivity of KRAS mutation CRC cells to metformin. The transcriptional level of MATE1 is decreased and the concentration of metformin in tumor cells is increased by upregulating the transmethylase DNMT1 and down-regulating the demethylase TET1/2, and thereby the inhibiting effect of metformin on tumor cell proliferation is enhanced. The present invention provides a novel thinking and method for the treatment of colorectal cancer, which has profound significance and is worth popularizing energetically.

### BRIEF DESCRIPTION OF THE DRAWINGS

FIG. 1 is a schematic diagram showing that clinical patients are divided into groups.

FIG. 2 shows that overall survival of colorectal cancer patients with diabetes mellitus is improved by metformin.

FIG. 3 shows that the overall survival of KRAS mutation colorectal cancer patients and the progression free survival during first-line chemotherapy are improved by metformin.

FIG. 4 is a diagram showing an intracellular gene modification mode in a SW48 KRAS (G13D) cell line.

FIG. 5 is an alignment diagram showing that KRAS exon2 GGC>GAC mutant type is positive cloning sequence.

FIG. 6 shows that the growth of KRAS mutation colorectal cancer cells is inhibited by metformin.

FIG. 7 shows that the growth of KRAS mutation PDX tumor is inhibited by metformin.

FIG. 8 shows that metformin accelerates the stagnation of KRAS mutation colorectal cancer cells in G1 phase to inhibit the proliferation of tumor cells.

FIG. 9 is a mechanism showing that metformin accelerates the stagnation of KRAS mutation colorectal cancer cells in G1 phase to inhibit the proliferation of tumor cells.

FIG. 10 shows that KRAS mutation colon cancer cells are more sensitive to metformin than KRAS wildtype colon cancer cells.

FIG. 11 shows that the concentration of metformin is accumulated in KRAS mutation colorectal cancer cells and PDX tumor tissues.

FIG. 12 shows that MATE1 is down-regulated in KRAS mutation colorectal cancer cells to improve the concentration of metformin in cells, thus enhancing the inhibiting effect of metformin on the proliferation of colorectal cancer cells.

FIG. 13 shows that MATE1 methylation is regulated in the KRAS mutation colorectal cancer cells to down-regulate the expression of MATE1.

FIG. 14 shows MATE1 promoter CpG island amplification sequences and primers after a genome DNA sample is modified by bisulfate.

FIG. 15 shows the expression of DNMT/TET in SW48 KRAS (G13D) cells and Lovo cells with knock-down KRAS.

FIG. 16 shows that the expression of MATE1 is down-regulated by regulating DMNT1/TET in the KRAS mutation colorectal cancer cells.

### DESCRIPTION OF THE EMBODIMENTS

The present invention will be further described in detail by reference to the accompanying drawings and detailed examples of the description; the examples described herein are merely illustrative of the present invention, but not constructed as limiting the protection scope of the present invention. Unless otherwise specified, the test methods used in the following examples are conventional methods; the materials, reagents and the like used may be purchased via business approaches.

### I. Experimental Materials

Fresh Tumor Tissue

Tumors in situ were excised from colorectal cancer patients who were confirmed, received and treated in Sun Yat-sen University Cancer Center (SYSUCC), and then about 5×5×5 mm cancer tissues were taken. The collection of cases should observe strictly the operation procedures of SYSUCC, and approved by the hospital and agreed by the patient and relations.

### Experimental Animals

4-6 week-old male nude mice (BALB/c nude mice, body weight: 14-18 g, purchased from Beijing Vital River Laboratory Animal Technology Co., Ltd., production license No.: SCXK (J.) 2016-0011)) were fed in a Specific Pathogen Free (SPF) environment of Sun Yat-Sen University (North Campus Experimental Animal Center); the usage license No. of the experimental unit is: SYXK (Y.) 2017-0081. The mice were subjected to experiments after passing quarantine inspection. Five basic welfares of the experimental animals were ensured in the feeding process, and the experimental process followed the 3R principle of Replacement, Reduction and Refinement.

Cell Line

SW48 was purchased from Shenzhen Otwo Biotechnology Co. Ltd; CaCO2, HCT-116 and LoVo were presented by The Sixth Affiliated Hospital Gastroenteropathy Research Institute of Sun Yat-Sen University; SW480 and SW620 cells were cell lines preserved in laboratory. STR identification of these 6 colorectal cancer cell lines were completed by Guangzhou CELLCOOK, and 100% matched with the information provided by ATCC, free of other cell contamination and STR alteration. KRAS genotypes of the above cells were inquired from the official website of ATCC and verified by PCR sequencing.

### II. Experimental Method

1. Shooting, Tumor Pathological Grading and Proliferative Cell Quantification

1) The overall tissue sample was shoot (10× and 20×) by an auto-digital slide scanning system (Axio Scan Z1).

2) After exporting images, the tumor was subjected to pathological grading by a pathologist, and divided into well differentiated, moderately differentiated, poorly differentiated and un-differentiated.

3) For the images of the Ki67-stained tumor tissues, an ImageJ IHC Profiler plug-in was used for quantitative analysis on the cell nucleus of Ki67(+), and denoted as proliferating-phase cells.

2. Detection of Cell Viability (a CCK-8 Method)

The cells were inoculated on a plate according to the concentration of 5000 cells/well (48-well plate, 200 μl per well). The cells were treated with different ways according to the experimental requirements, and 10 μl CCK8 solution was added after a period of time. The cells were incubated for 1 h in an incubator; 200 μl supernatant were absorbed into a 96-well ELISA plate to determine OD₄₅₀ with a microplate reader.

### Example 1 Influences of Metformin on the Prognosis of Metastatic Colorectal Cancer (mCRC) Patients

I. Grouping of mCRC Patients with T2DM and Clinical Characteristics

### 1. Experimental Sample

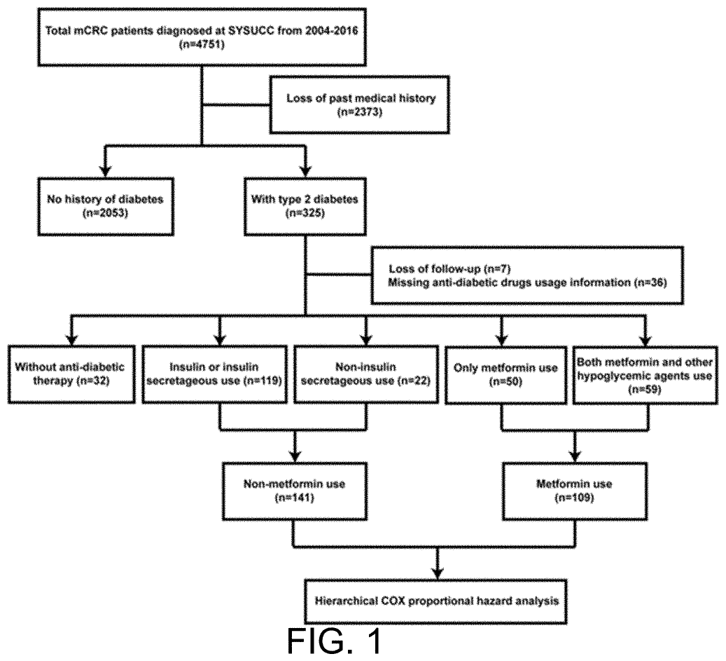

282 mCRC patients suffering from type 2 diabetes mellitus (T2DM) before being confirmed were brought from 4751 metastatic colorectal cancer (mCRC) patients received and treated in SYSUCC from 2004 to 2016, and divided into group (metformin use, n=109), group (insulin or insulin-releasing, n=141), group (other anti-diabetic drugs use, n=22) and group (without anti-diabetic treatments, n=32). Grouping of the patients is shown in FIG. 1.

### 2. Experimental Method

General clinical characteristics of the patients, such as, sex, age and body mass index (BMI) and the clinical characteristics affecting mCRC prognosis in the existing research reports (such as, primary tumor site, pathological grading, metastatic site, first-line chemotherapy regimen and KRAS genotype) were collected to exclude confounding factors, thus making the influences of metformin on the overall survival (OS) of the mCRC patients with T2DM and the progression free survival (PFS) of the first-line chemotherapy clear. Basic clinical characteristic information of the patients is shown in Table 1.

Table 1. Basic clinical information of mCRC patients with T2DM

TABLE 1
Baseline characteristics of mCRC patients with T2DM,
according to hypoglycemic agents use, SYSUCC 2004-2016

| | | Hypoglycemic agents use | |
|---|---|---|---|
| | Total No. | Nonusers | Users |
| Characteristics | (%) | No. (%) | No. (%) |
| Sex | | | |
| Male | 208 (73.76) | 25 (78.13) | 183 (73.20) |
| Female | 74 (26.24) | 7 (21.88) | 67 (26.80) |
| Age at diagnosis, | | | |
| years | | | |
| <60 | 93 (32.98) | 9 (28.13) | 84 (33.60) |
| ≥60 | 189 (67.02) | 23 (71.88) | 166 (66.40) |
| BMI (kg/m²) | | | |
| Lean (<18.5) | 16 (5.67) | 1 (3.13) | 15 (6.00) |
| Normal (18.5~23.0) | 154 (54.61) | 15 (46.88) | 154 (54.61) |
| Pre- & obese (>23.0) | 112 (39.72) | 16 (50.00) | 112 (39.72) |
| Primary site | | | |
| Right colon | 62 (21.99) | 8 (25.00) | 54 (21.60) |
| Left colon | 109 (38.65) | 8 (25.00) | 101 (40.40) |
| Rectum | 111 (39.36) | 16 (50.00) | 95 (38.00) |
| Pathological grading | | | |
| Well differentiated | 7 (2.53) | 0 (0.00) | 7 (2.86) |
| Moderately | 177 (63.90) | 20 (62.50) | 157 (64.08) |
| differentiated | | | |
| Poorly & un- | 93 (33.57) | 12 (37.50) | 81 (33.06) |
| differentiated | | | |
| Unknown | 5 | 0 | 5 |
| Metastatic site | | | |
| Liver | 122 (43.26) | 12 (37.50) | 110 (44.00) |
| Other organs | 43 (15.25) | 4 (12.50) | 39 (15.60) |
| Distant lymph node | 28 (9.93) | 6 (18.75) | 22 (8.80) |
| Multiple metastatic | 54 (19.15) | 3 (9.38) | 51 (20.40) |
| sites | | | |
| Peritoneum | 35 (12.41) | 7 (21.88) | 28 (11.20) |
| KRAS genotype | | | |
| Wildtype | 127 (60.19) | 10 (52.63) | 117 (60.94) |
| Mutation | 84 (39.81) | 9 (47.37) | 75 (39.06) |
| Unknown | 71 | 13 | 58 |

Abbreviations: mCRC, metastatic colorectal cancer; T2DM, type 2 diabetes mellitus; BMI, body mass index.

Whether there exists a difference in the distribution of individual's clinical characteristics between the group (metformin use) and other non-metformin use groups was subject to statistics. Continuous variables (age and BMI) were analyzed by a single-factor method, and classified variables (sex, primary tumor site, pathological grading, KRAS genotype, metastatic site, first-line chemotherapy regimen, age grouping and BMI grouping) were subjected to chi-square test.

### 3. Experimental Results

The 180 patients were subjected to statistical analysis to indicate that there is no statistic difference (P>0.05) in sex, age, BMI, primary tumor site, pathological grading, metastatic site, and KRAS genotype between the metformin use and other non-metformin use groups, as shown in Table 2.

Table 2. Distribution of clinical characteristics of the 180 cases of mCRC patients with T2DM having a confirmed Kras genotype in metformin use or other hypoglycemic agents use

TABLE 2
List of the probability distribution of mCRC patients characteristics
with metformin use or other hypoglycemic agents use

| | | Groups | | |
|---|---|---|---|---|
| | | | Other | |
| | | | hypoglycemic | |
| | Total No. | Metformin | agents | P |
| Characteristics | (%) | use No. (%) | use No. (%) | value |
| Sex | | | | 0.511 |
| Male | 133 (73.89) | 64 (76.19) | 69 (71.88) | |
| Female | 47 (26.11) | 20 (23.81) | 27 (28.12) | |
| Age at diagnosis, | | | | 0.495 |
| years | | | | |
| <60 | 66 (36.67) | 33 (39.29) | 33 (34.38) | |
| ≥60 | 114 (63.33) | 51 (60.71) | 63 (65.63) | |
| BMI (kg/m^{2) a} | | | | 0.544 |
| Normal (18.5~23.0) | 105 (58.33) | 51 (60.71) | 54 (56.25) | |
| Pre- & obese (>23.0) | 75 (41.67) | 33 (39.29) | 42 (43.75) | |
| Primary site | | | | 0.069 |
| Right colon | 43 (23.89) | 15 (17.86) | 28 (29.17) | |
| Left colon | 78 (43.33) | 35 (41.67) | 43 (44.79) | |
| Rectum | 59 (32.78) | 34 (40.48) | 25 (26.04) | |
| Metastatic site | | | | 0.133 |
| Liver | 81 (45.00) | 35 (41.67) | 46 (47.92) | |
| Other organs | 31 (17.22) | 20 (23.81) | 11 (11.46) | |
| Distant lymph node | 12 (6.67) | 7 (8.33) | 5 (5.21) | |
| Multiple metastatic | 34 (18.89) | 15 (17.86) | 19 (19.79) | |
| sites | | | | |
| Peritoneum | 22 (12.22) | 7 (8.339) | 15 (15.63) | |
| Pathological grading | | | | 0.658 |
| Well differentiated | 4 (2.22) | 1 (1.20) | 3 (3.12) | |
| Moderately | 112 (62.22) | 52 (61.90) | 60 (62.50) | |
| differentiated | | | | |
| Poorly & un- | 64 (35.56) | 31 (36.90) | 33 (34.38) | |
| differentiated | | | | |
| KRAS genotype | | | | 0.237 |
| Wildtype | 109 (60.56) | 47 (55.95) | 62 (64.58) | |
| Mutation | 71 (39.44) | 37 (44.05) | 34 (35.42) | |

Abbreviations: mCRC metastatic colorectal cancer; T2DM, type 2 diabetes mellitus; BMI, body mass index.
Note:
^{a}. 10 lean patients (BMI < 18.5) were all other hypoglycemic agents' user, so this characteristic is inappropriate for hierarchical COX proportional hazards analysis.

### II. Metformin Improves the OS of mCRC Patients with T2DM and PFS of the First-Line Chemotherapy

A Kaplan-Meier curve was used to analyze and confirm whether metformin improved the prognosis of the CRC patients with T2DM in the center compared with other hypoglycemic agents use.

### 1. Experimental Method

GraphPad Prism 7 was used to draw a Kaplan-Meier curve, and Log-rank (Mantel-Cox) statistical analysis was performed; stratified HR was additionally used to make a statistics on the influences on OS and PFS between different hypoglycemic agents use and the without anti-diabetic treatments.

### 2. Experimental Results

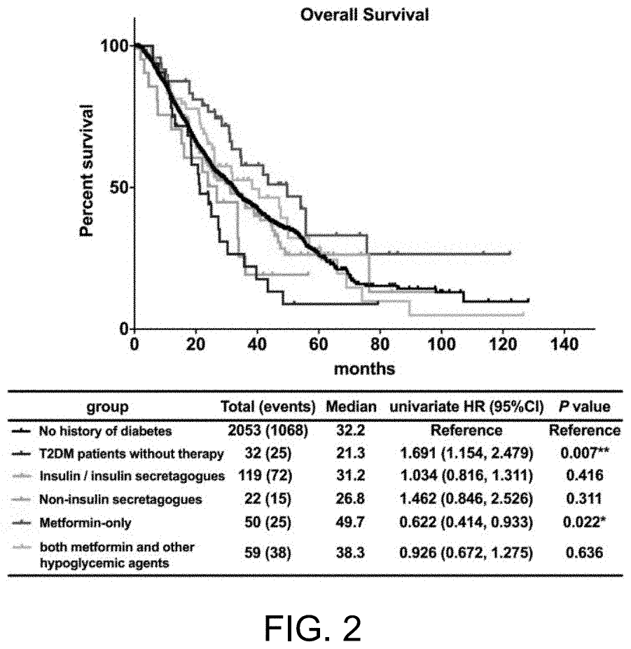

As shown in FIG. 2, the median survival of the without anti-diabetic treatments use decreases 11.2 months (P=0.007) compared with the group without T2DM, and the median survival of the metformin use extends 11.3 months (P=0.022), and the other hypoglycemic agents have no substantial improvement. The above result indicates that metformin may significantly extend the overall survival in the 282 cases of the mCRC patients with T2DM diagnosis confirmed from 2004 to 2016 in SYSUCC. In addition, the statistics of the stratified HR indicates that (see Table 3) the hypoglycemic therapy improves prognosis (HR=0.547, 95% CI: 0.327-0.913); the single use or combined use of metformin may improve the prognosis of mCRC patients, while there is no statistic difference in other hypoglycemic agents. Therefore, the result indicates that the improvement effect of metformin on mCRC prognosis may be further associated with other factors besides the reduction of the blood sugar.

Table 3. Influences on OS and PFS in different hypoglycemic agents use and without anti-diabetic treatments

TABLE 3
Association between different hypoglycemic agents use and OS and
PFS, SYSUCC 2004-2016

| | OS (n = 281) | | PFS (n = 208) | |
|---|---|---|---|---|
| Hypoglycemic | Total | HR | Total | HR |
| agents use | (events) | (95% CI)^{c} | (events) | (95% CI)^{c} |
| Nonusers | 32 (25) | Reference | 26 (21) | Reference |
| Any-hypoglycemic | 250 (150) | 0.547 (0.327, | 182 (157) | 1.031 (0.585, |
| agents use | | 0.913) | | 1.818) |
| Insulin or/and insulin | 119 (72) | 0.647 (0.374, | 91(82) | 1.129 (0.599, |
| secretagogues^{a} | | 1.118) | | 2.129) |
| Non-insulin | 22 (15) | 0.875 (0.397, | 12 (8) | 0.669 (0.209, |
| secretagogues^{b} | | 1.926) | | 2.142) |
| Metformin-only | 50 (25) | 0.356 (0.183, | 35 (29) | 0.630 (0.310, |
| | | 0.693) | | 1.280) |
| Both metformin and | 59 (38) | 0.491 (0.269, | 44 (38) | 0.811 (0.406, |
| other hypoglycemic | | 0.894) | | 1.621) |
| agents | | | | |

Abbreviations: OS, overall survival; PFS, progression-free survival; SYSUCC, Sun Yat-sen University Cancer Center; HR, hazard ratio.
Notes:
^{a}Insulin secretagogues include sulfonylureas (e.g. Gliclazide, Glipizide, Glimepiride) and non-sulfonylureas (e.g. Repaglinide, Nateglinide).
^{b}Non-insulin secretagogues include α-glucosidase inhibitors (e.g. Acarbose, Voglibose, Miglitol) and Thiazolidinediones (e.g. Rosiglitazone, Pioglitazone).
^{c}HR (95% CI) was stratified by age at diagnosis (<60, ≥60 years) and adjusted for sex, body mass index, primary tumor site, metastatic site, pathological grading, KRAS genotype.

### III. The Efficacy of Metformin on Patients' Death or Tumor Progression Accords with the Proportional Hazard Assumption

### 1. Experimental Method

Proportional hazard assumption was performed. That is, it is assumed that the efficacy of metformin on patients' death or tumor progression does not change with time, but a fixed value. Kolmogorov-Smirnov test and Cramer von Mises test were used for hypothesis testing.

### 2. Experimental Results

The result of Table 4 indicates that the efficacy of metformin on patients' death or tumor progression accords with the proportional hazard assumption (P>0.05).

TABLE 4
Proportional hazard assumption to the efficacy of metformin
on patients' death or tumor progression

| | Metformin: other hypoglycemic agents | | |
|---|---|---|---|
| | Inspect | | |
| Condition: | whether | changes with time | |
| death or | the effect | | |
| progression | H₀ | The effect does not change with time | |
| | Inspection | Kolmogorov- | Cramer von |
| | method | Smirnov inspection | Mises inspection |
| Time | | | |
| variable | | | |
| (P value) | | | |
| OS | | 0.898 | 0.593 |
| PFS | | 0.252 | 0.255 |

### IV. KRAS Mutation mCRC Patients Benefit from Metformin More Significantly

A stratified Cox proportional hazard model was used for stratification on the above collected clinical characteristics to explore the individual factors influencing the efficacy of metformin.

### 1. Experimental Method

After through the proportional hazard assumption, it is further assumed that sex, age, BMI, primary tumor site, pathological grading, metastatic site, and KRAS genotype may serve as confounding factors to make an impact on the anti-tumor effect of metformin. Therefore, hierarchical regression analysis was performed on the above clinical characteristics, thus determining the individual difference interacting with metformin.

### 2. Experimental Results

As shown in Table 5, after the above clinical characteristics were brought into the equation of proportional hazard regression model as confounding factors, it can be seen that compared with the use of other hypoglycemic agents, metformin has a hazard ratio (HR) of reducing death of 0.746, 95% confidence interval (CI) of 0.496-1.121, interval span of 1, and there is no statistic difference (P>0.05); the HR of reducing tumor progression during first-line chemotherapy is 0.737, and 95% CI is 0.501-1.086, and there is no statistic difference (P>0.05), either, indicating that there is an individual difference on whether mCRC patients benefit from metformin compared with other hypoglycemic agents.

The above clinical characteristics were subjected to hierarchical regression analysis. The result shows that for the KRAS mutation mCRC patients, metformin may reduce the risk of death (HR=0.272, 95% CI is 0.120-0.617) better than other hypoglycemic agents, and may also reduce the risk of tumor progression during first-line chemotherapy (HR=0.405, 95% CI is 0.212-0.774). Moreover, R language-based EmpowerStats software was used for interaction inspection to find that KRAS mutation may significantly enhance the effect of metformin on reducing mortality risk (P_{interaction}<0.001) and the effect on reducing the risk of tumor progression during first-line chemotherapy (P_{interaction}=0.02). Furthermore, Kaplan-Meier curve was used to respectively analyze KRAS wildtype and KRAS mutation mCRC patients to find that metformin significantly extends the overall survival (P<0.001) of the KRAS mutation mCRC patients and the progression free survival (P<0.01) of first-line chemotherapy (FIGS. 3A-B), while metformin has no efficacy in KRAS wildtype mCRC patients (FIGS. 3C-D).

Table 5 Analysis on the proportional hazard regression model of metformin with OS and PFS and interaction between each clinical characteristic

TABLE 5
Association between metformin use and OS and PFS, according to the
characteristics of mCRC patients with T2DM, SYSUCC 2004-2016

| | OS | | | PFS | | |
|---|---|---|---|---|---|---|
| | No. of | HR (95% | | No. of | HR (95% | |
| Characteristics ^{a} | events ^{b} | CI) | P_{interaction} | events ^{c} | CI) | P_{interaction} |
| Metformin use vs | 51:68 | 0.746 (0.496, | | 55:60 | 0.737 (0.501, | |
| non-metformin | | 1.121) | | | 1.086) | |
| use | | | | | | |

Metformin use vs. non-metformin use after stratifying with

| | | | | | | |
|---|---|---|---|---|---|---|
| Sex | | | 0.321 | | | 0.493 |
| Male | 37:44 | 0.673 (0.401, | | 42:43 | 0.732 (0.468, | |
| | | 1.131) | | | 1.146) | |
| Female | 14:15 | 0.944 (0.385, | | 13:17 | 0.697 (0.318, | |
| | | 2.318) | | | 1.529) | |
| Age at diagnosis, | | | 0.697 | | | 0.054 |
| years | | | | | | |
| <60 | 21:19 | 0.869 (0.411, | | 23:22 | 1.074 (0.580, | |
| | | 1.715) | | | 1.990) | |
| ≥60 | 30:40 | 0.596 (0.348, | | 32:38 | 0.562 (0.339, | |
| | | 0.988) | | | 0.930) | |
| BMI (kg/m²) | | | 0.867 | | | 0.430 |
| Normal | 33:33 | 0.582 (0.334, | | 37:33 | 0.884 (0.542, | |
| (18.5~23.0) | | 1.013) | | | 1.442) | |
| Pre- & obese | 18:26 | 0.702 (0.357, | | 18:27 | 0.555 (0.287, | |
| (>23.0) | | 1.382) | | | 1.073) | |
| Primary site | | | 0.444 | | | 0.426 |
| Right colon | 10:17 | 0.729 (0.294, | | 8:15 | 0.622 (0.255, | |
| | | 1.807) | | | 1.517) | |
| Left colon | 21:24 | 0.982 (0.527, | | 22:29 | 0.823 (0.451, | |
| | | 1.830) | | | 1.500) | |
| Rectum | 20:18 | 0.475 (0.215, | | 25:16 | 0.544 (0.282, | |
| | | 1.051) | | | 1.048) | |
| Metastatic site | | | — | | | — |
| Liver | 23:25 | 1.151 (0.563, | | 23:29 | 0.949 (0.481, | |
| | | 2.354) | | | 1.871) | |
| Other organs | 9:4 | — | | 10:4 | — | |
| Distant lymph | 7:5 | — | | 4:2 | — | |
| nodes | | | | | | |
| Multiple sites | 10:12 | 1.008 (0.346, | | 14:16 | 0.818 (0.329, | |
| | | 2.939) | | | 2.031) | |
| Peritoneum | 2:13 | — | | 4:9 | — | |
| Pathological | | | — | | | — |
| grading | | | | | | |
| Well- | 1:0 | — | | 0:2 | — | |
| differentiated | | | | | | |
| Moderately | 33:38 | 0.676 (0.407, | | 32:35 | 0.644 (0.384, | |
| differentiated | | 1.125) | | | 1.080) | |
| Poorly & un- | 17:21 | 0.629 (0.245, | | 23:23 | 0.811 (0.445, | |
| differentiated | | 1.616) | | | 1.477) | |
| KRAS genotype | | | <0.001 | | | 0.020 |
| Wildtype | 32:37 | 1.487 (0.844, | | 32:39 | 1.194 (0.720, | |
| | | 2.620) | | | 1.979) | |
| Mutation | 19:22 | 0.272 (0.120, | | 23:21 | 0.405 (0.212, | |
| | | 0.617) | | | 0.774) | |

Abbreviations:
OS, overall survival; PFS, progression-free survival; mCRC, metastatic colorectal cancer; T2DM, type 2 diabetes mellitus; SYSUCC, Sun Yat-sen University Cancer Center; HR, hazard ratio; BMI, body mass index; P_{interaction}, P value for interaction test.
Notes:
^{a} Each characteristic was stratified by age at diagnosis and adjusted for the other variables listed above.
^{b} The number of death cases after diagnosis as mCRC.
^{c} The number of cases dead or getting worse during first-line chemotherapy.
“—” indicates the variable is excluded because of <5 observations in this category.

### V. Metformin Inhibits the Proliferation of KRAS Mutation CRC Cells

The effect of the clinical characteristic on the anti-tumor treatment with metformin was verified by immunohistochemistry and cytology experiments of the colorectal cancer tissue sections.

### 1. Experimental Method

(1) To further make the effect of metformin on KRAS mutation CRC cells clear, pathological sections (including a primary lesion and a metastasis lesion) from 28 cases of patients who were diagnosed with metastatic colorectal cancer and had taken metformin before tumor excision were collected, and DNA of paraffin-embedded tissues was extracted to identify the KRAS genotype.

Hematoxylin-eosin stain (H&E) was used to judge the position of tumor cells (cell nucleus was darkly stained, heterogeneous, and glandular epithelium structure was broken), and then cells in the proliferating phase were marked by Ki67 staining.

\textcircled{1} H&E

1) Immobilized tumor tissues were made into paraffin sections;

2) the paraffin sections were dewaxed with xylene;

3) the paraffin sections were put to methanol for immobilization for 2 min;

4) hematoxylin stain was performed for 3 min;

5) color separation solution: 70% ethyl alcohol+glacial acetic acid 10 ml (for several minutes until an appropriate color);

6) Bluing in running tap water within 5 minutes;

7) eosin stain was performed for 1 min;

8) the sections were treated by 75%, 80%, 90% and 95% absolute ethyl alcohol respectively for 30 sec;

9) the sections were treated by 2-3 cylinders of 100% ethyl alcohol, 30 sec for each cylinder;

10) the sections were treated by 3 cylinders of 100% xylene, the first cylinder for 10 min, second and third cylinders for 5 min;

11) the sections were sealed with a neutral gum.

\textcircled{2} Ki67 stain

1) the sections were dried at 65° C. for 2 h;

2) the sections were dewaxed with xylene for 30 min×3 times at room temperature;

3) the sections were treated with absolute ethyl alcohol for 10 minx 1 times at room temperature;

4) the sections were hydrated with 100%, 95%, 90%, 80% and 70% ethyl alcohol respectively for 5 min;

5) the sections were washed with dH₂O for 5 minx 1 times;

6) the sections were washed with PBST for 5 min×3 times;

7) antigen repair: after 1 L of 10 mmol/L citrate buffer solution (PH=6.0) was put to a pressure cooker to be boiled, and the sections were placed to the pressure cooker and immersed completely, and the cooker cover was screwed until air discharge, and time was controlled for 2 min, then the buffer solution was naturally cooled till room temperature, and the sections were taken out;

8) the sections were washed with dH₂O for 2 min×2 times;

9) the sections were washed with PBST for 5 min×3 times;

10) occlusion: the sections were encircled with an immunohistochemical pen, and 3% H2O2 was added for 30 min at room temperature;

11) the sections were washed with PBST for 5 min×3 times;

12) blocking: the sections were blocked with goat serum for 1 h at room temperature, and then washed for 5 min×3 times with PBST;

13) primary antibody incubation: Ki67 was diluted by an antibody diluent according to 1:400, and covered on the tissues; then the sections were placed to a wet box to prevent dry, standing over the night at 4° C.;

14) the sections were washed with PBST for 5 min×3 times;

15) second antibody: an A solution of a universal (mice/rabbit) immunohistochemical detection kit, the sections were incubated for 30 min at room temperature;

16) the sections were washed with PBST for 5 min×3 times;

17) DAB developing: the sections were diluted with a B solution of a universal (mice/rabbit) immunohistochemical detection kit according to a ratio of 1:50 into a C solution, and after the reaction was stopped with tap water, the sections were washed with water for 30 min;

18) the sections were re-stained by hematoxylin for 1 min, and differentiated by hydrochloric acid alcohol (1:1000) for 10 s to remove non-specific coloring; then after the reaction was stopped with tap water, the sections were washed with water for 20 min;

19) the sections were hydrated with 70%, 80%, 90%, 95% and 100% ethyl alcohol respectively for 5 min;

20) and the sections were dehydrated with xylene to be transparent for 5 min×2 times, and then sealed with a neutral gum.

(2) Metformin with a gradient concentration was used in vitro to treat KRAS wildtype colorectal cancer cell lines SW48 and CaCO2, KRAS G13D-mutation colorectal cancer cell lines HCT-116 and LoVo, as well as KRAS G12V-mutation colorectal cancer cell lines SW480 and SW620. In addition, the metformin with a gradient concentration was used to treat the KRAS wildtype colorectal cancer cell line SW48 transfected with KRAS G12V, KRAS G13D and KRAS G12D plasmids.

(3) Metformin with a gradient concentration was used in vitro to treat the KRAS (G13D)-mutation cell line SW48 constructed via a CRISPR/Cas9 system, which forward verifies that metformin inhibits the viability of the KRAS mutation colorectal cancer cells.

\textcircled{1} Construction of a KRAS G13D Point Mutation CRISPR/Cas9 Plasmid

1) sgRNA (sequence of guide RNA) design: the design was performed according to bases on the former 250 bp of initiation codon ATG of No. 2 exon of the KRAS gene on human No. 12 chromosome on the website http://crispr.mit.edu provided by MIT in Zhang Lab. sgRNA with lower off-target efficiency (score>85) was taken and website http://www.rgenome.net/cas-offinder/of CRISPR RGEN tool Cas-OFFinder was used to configure the number of mismatched bases ≤2; sgRNA having mismatched bases was not used to avoid the nonspecific cutting. sgRNA: 5′-GCATTTTTCTTAAGCGTCGA-3′ was selected.

2) gRNA Synthesis and Binding to a Recombinant Vector:

A. gRNA oligo (PAGE purification) was synthesized by a chemical method; the underline represents Bbsl enzyme-cutting binding site; 3′-terminal of the reverse sequence is not to add a base on cytimidine, but as follows:

Target sequence (PAM):
GCATTTTTCTTAAGCGTCGA (TGG);
Forward:
CACCGCATTTTTCTTAAGCGTCGA;
Reverse:
AAACTCGACGCTTAAGAAAAATGC.

B. Each OD of oligo was dissolved by ddH₂O (ratio of 10 μL/1 nmol) with a final concentration of 100 μM. The oligo was synthesized into double-stranded nucleotide by the following reaction:

Reaction system (10 μL)

| | |
|---|---|
| Oligo forward | 1 μL |
| Oligo reverse | 1 μL |
| 10× T4 Ligation Buffer | 1 μL |
| T4 PNK | 0.5 μL |
| ddH₂0 | 6.5 μL |

Reaction conditions

| Temperature | Time | Cycle |
|---|---|---|
| 37° C. | 30 min | 1 cycle |
| 95° C. | 5 min | 15 cycles (−5° C./cycle) |
| 10° C. | ∞ | |

C. Enzyme digestion: a Bbs I restriction enzyme was used for the digestion of a pSpCas9(BB)-2A-puro (PX459) V2.0 plasmid, then agarose electrophoresis and gel recovery and purification were performed. Enzyme digestion conditions were as follows:

Reaction system (60 μL) and reaction for 15 min at 37° C.

| | |
|---|---|
| FastDigest Bpil | 3 μL |
| PX459 | 3 μg |
| 10× FastDigest Buffer | 6 μL |
| ddH₂O | To 60 μL |

D. Linking: PX459 and gRNA were linked by a T4 ligase, and then agarose electrophoresis identification and gel recovery and purification were performed, denoted as PX459/hKRAS gRNA. Linking conditions were as follows:

Reaction system (20 μL) and reaction for 10 min at 37° C.

| | | |
|---|---|---|
| gRNA (diluted according to 1:250) | 2 | μL |
| PX459 (purification after enzyme digestion) | 100 | ng |
| 2× Quick Ligation Buffer | 10 | μL |
| Quick Ligase | 2 | μL |

| | |
|---|---|
| ddH₂O | To 20 μL |

3) Construction of a KRAS G13D Point Mutation Donor Plasmid:

A. Gene retrieval: genome DNA of LoVo was extracted by a genome DNA extraction kit first, and then amplified into about 3000 bp DNA sequence containing sgRNA and exon2 by a high-fidelity PCR method, subjected to electrophoresis identification, gel recovery and purification. PCR reaction system and conditions were as follows:

Reaction system (50 μL)

| | |
|---|---|
| 2× PrimeSTAR Max Premix | 25 μL |
| hKRAS-Homology-F | 1 μL |
| hKRAS-Homology-R | 1 μL |
| DNA template | 2 μL |
| ddH₂O | To 50 μL |

Reaction conditions

| Process | Temperature | Time | Cycle |
|---|---|---|---|
| Denaturation | 98° C. | 10 s | 35 cycle |
| Annealing | 55° C. | 5 s | |
| Extension | 72° C. | 45 s | |
| Preservation | 4° C. | ∞ | |

B. T vector construction: the DNA purified in the above step was subjected to A addition reaction, and linked onto a pGM-T vector, and transferred into competent cells for amplification, then monoclonal antibodies were picked for sequencing verification. If the sequence was verified correct, it was denoted as pGM-T/KRAS-homology. A addition reaction and ligation reaction conditions were as follows:

A addition reaction system and incubation
for 30 min at 72° C.

| | |
|---|---|
| DNA | 15 μL |
| Tailing-A Reaction Buffer | 4 μL |
| Taq DNA Polymerase (2.5 U/μL) | 1 μL |

Ligation reaction system (10 μL) and reaction for
5 min at room temperature

| | | |
|---|---|---|
| 2× RapiLigation Mix | 5 | μL |
| pGM-T Vector (50 ng/μL) | 1 | μL |
| DNA | 200 | ng |

| | |
|---|---|
| ddH₂O | To 10 μL |

C. PAM of the sgRNA was subjected to TGG>TGA point mutation: the sequence in front of sgRNA PAM TGG was amplified by a high-fidelity PCR method, where primers were hKRAS-Left arm-F and hKRAS-TGG mut-Left arm-R; a 15 bp sequence homologous to a broken terminal pGM-T 3′ was introduced in the forward; a point mutation sequence CCA>TCA was introduced at the 5′ terminal of the reverse primer; primers of another DNA fragment were hKRAS-TGG mut-Right arm-F and hKRAS-Right arm-R; a 15 bp sequence (including ACC>ACT) homologous to 3′ terminal of the former DNA fragment was introduced on the 5′ terminal of the forward primer, and a sequence homologous to a broken terminal pGM-T 5′ was introduced in the 3′ terminal. PCR reaction system of the PrimeSTAR Max Premix was the same as the above, and reaction condition: extension time was changed to 15 s. After the 2 PCR products were recovered and purified by a gel, the 2 PCR products were linked with a pGM-T vector by seamless cloning, and then subjected to gel recovery and purification, transformation, amplification; then monoclonal antibodies were picked for sequencing. Seamless cloning reaction system and conditions were as follows:

Reaction system (20 μL) and reaction for 30 min at 37° C.,
transferring to an ice bath rapidly for 5 min

| | | |
|---|---|---|
| 5× CE MultiS Buffer | 4 | μL |
| pGM-T vector (50 ng/μL) | 1.2 | μL |
| hKRAS-Right arm | 30 | ng |
| hKRAS-Left arm | 30 | ng |
| Exnase MμltiS | 2 | μL |

| | |
|---|---|
| ddH₂O | To 20 μL |

D. The sequence for homologous recombination was linked onto a donor vector pDONR 221: attB1 was introduced at the 5′ terminal and 3′ terminal respectively by primer design, then fragments were linked on the pDONR 221 by BP reaction to form Entry Clone, denoted as a pDONR 221/hKRAS-homology plasmid. Primer design is shown in Table 6.

TABLE 6
Naming and corresponding sequences of the primers used

| | Sequences (5′ to 3′); the underline denotes a homologous arm |
|---|---|
| Primer name | sequence, and the lower case denotes point mutation bases |
| hKRAS-Homology-F | TAAACATTCATATTATTTCAAAAAATTTGAAAC |
| (SEQ ID NO: 1) | |
| hKRAS-Homology-R | TGCCCTCAGAACTTGCCTCAGCGC |
| (SEQ ID NO: 2) | |
| hKRAS-Left arm-F | ATTCGTATCCAAGATTAAACATTCATATTATTTCAAAA |
| (SEQ ID NO: 3) | AATTTGAAAC |
| hKRAS-TGG mut- | tCATCGACGCTTAAGAAAAATGCATAAATGCT |
| Left arm-R | |
| (SEQ ID NO: 4) | |
| hKRAS-TGG mut- | GAATTCGCAGCTACtAGGAGTTTGTAAATGAAGTACAG |
| Right arm-F | TT |
| (SEQ ID NO: 5) | |
| hKRAS-Right arm-R | TAGTGTATCCAAGTATGCCCTCAGAACTTGCCTCAGCG |
| (SEQ ID NO: 6) | CA |
| hKRAS gRNA-F- | CACCGCATTTTTCTTAAGCGTCGA |
| oligo | |
| (SEQ ID NO: 7) | |
| hKRAS gRNA-R- | AAACTCGACGCTTAAGAAAAATGC |
| oligo | |
| (SEQ ID NO: 8) | |
| hKRAS-attB1 | GGGGACAAGTTTGTACAAAAAAGCAGGCTTAAACATTC |
| (SEQ ID NO: 9) | ATATTATTTCAAAAAATTTGAAAC |
| hKRAS-attB1 | GGGGACCACTTTGTACAAGAAAGCTGGGTTGCCCTCAG |
| (SEQ ID NO: 10) | AACTTGCCTCAGCGCA |
| hKRAS-OE-F | TGGAATTCTGCAGATGCCACCATGACTGAATATAAACT |
| (SEQ ID NO: 11) | TG |
| hKRAS-OE-R | GCCACTGTGCTGGATTTACATTATAATGCATTTTT |
| (SEQ ID NO: 12) | |

pDONR 221 linking reaction system and conditions were as follows:

Reaction system (20 μL) and reaction for 60
min at room temperature

| | | |
|---|---|---|
| pDONR 221 (150 ng/μL) | 2 | μL |
| hKRAS-homology | 100 | ng |
| BP Clonase ™ II | 2 | μL |

| | |
|---|---|
| ddH₂O | To 20 μL |

\textcircled{2} Construction of a SW48 KRAS (G13D) Cell Line

1) SW48 was subcultured to a 3 cm³ culture dish, when the degree of cell fusion was up to 80%-90%, fresh culture solution was replaced ahead of 2 h. 1.5 μg PX459/hKRAS gRNA and 1.5 μg pDONR 221/hKRAS-homology plasmids were respectively taken and added to 150 μL opti-MEM, and 9 μL P3000 was added; 3.5 μL Lipo3000 was separately taken and added to 150 μL opti-MEM, and incubated for 5 min at room temperature. A premixed solution of the plasmid was added to a Lipo3000 premixed solution, and incubated for 15 min at room temperature, and then SW48 cell culture solution was dropwise added.

2) After the cells were transfected for 12 h, fresh culture solution was replaced, 1 μM Scr7 was added to inhibit the reaction of NHEJ, thus improving the efficiency of homologous recombination (4-5 times may be improved according to literature reports). After being transfected for 24 h, 1 μM puromycin was added to screen positive-transfection cells.

3) After being transfected for 72 h, fresh culture solution was replaced to thoroughly remove puromycin. If the degree of cell fusion was 70%-90%, cell vitality was better, and then the cells were subcultured.

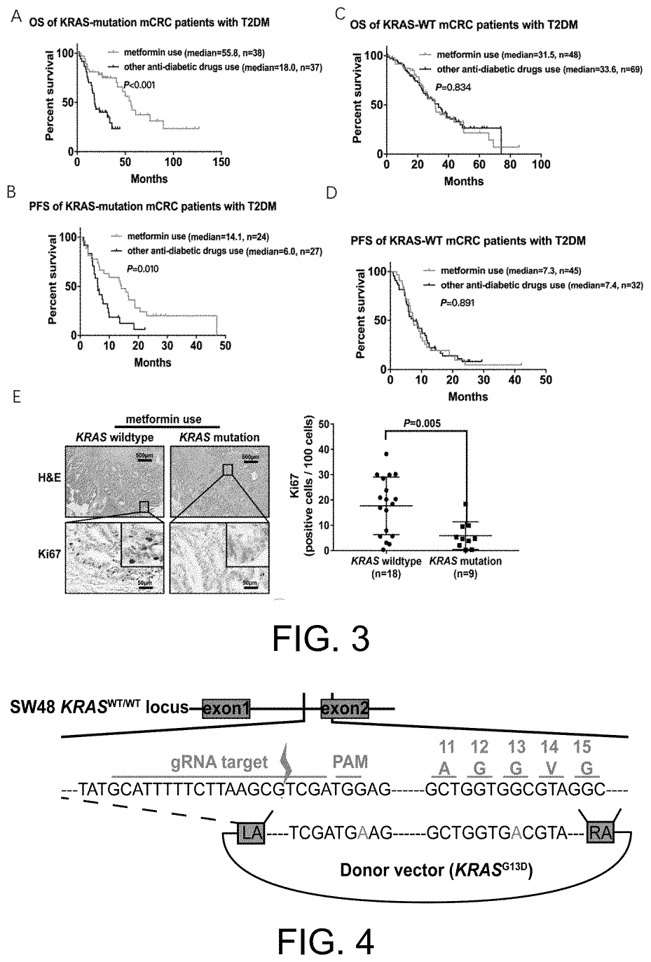

4) After being subcultured, 1×10⁶ cells were taken to extract genome DNA, hKRAS-Homology-F and hKRAS-Homology-R were used for PCR amplification; the steps and reaction conditions were the same as the above. The cells were linked to pGM-T only and transformed into DH5α for amplification, the cells were smeared on the plate, and about 30 clones were picked for sequencing. If there are sgRNA PAM TGG>TGA and KRAS exon2 GGC>GAC mutations in the sequencing result, it indicates that homologous recombination is successful. The diagram of an intracellular gene modification mode is shown in FIG. 4.

5) The cells were digested and resuspended in case of ensuring a better cell viability of the mixed SW48 cell line, 200 cells were counted to regulate the concentration to 1 cell/200 μL, and inoculated on a 96-well plate, there was about 1 cell in 200 μl culture solution per well.

6) The cells were observed every other day to note the humidity of the incubator, thus preventing the evaporation of the culture solution in the 96-well plate. After a single cell was cloned, 20-50 clones were picked out for subculture.

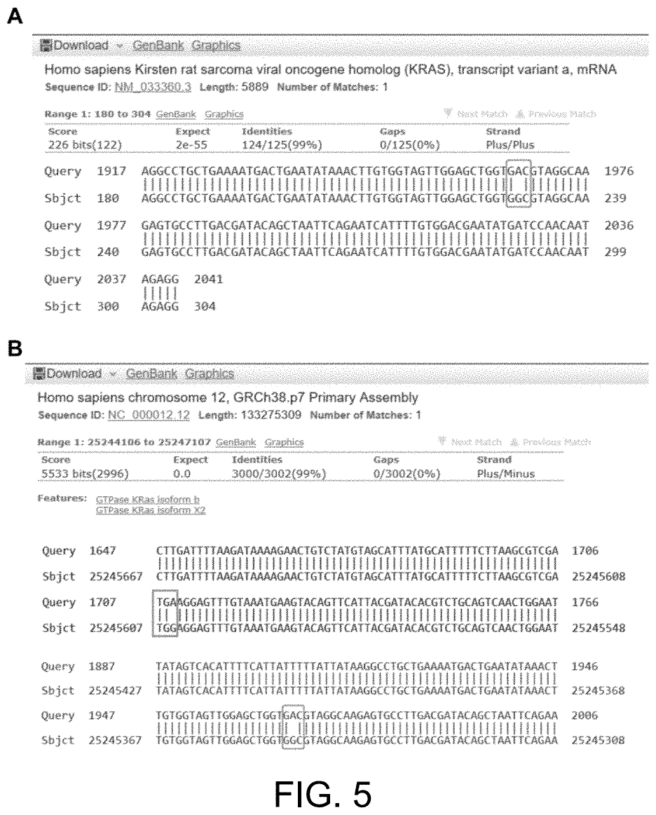

7) After the monoclonal cell line was amplified, the operation was the same as the above. RNA was extracted from each cell clone, and were converted to cDNA, KRAS was amplified, transformed and conjugated into T vector to make a library, and then a bacterial clone was sent for sequencing. The presence of KRAS exon2 GGC>GAC mutant type is called positive clone. Breed conservation was performed and denoted as a SW48 KRAS (G13D) cell. The positive sequence alignment is shown in FIG. 5.

(4) Metformin with a gradient concentration was used in vitro to treat lentivirus shRNA to construct a LoVo cell line with knock-down KRAS, which reversely verifies that metformin inhibits the viability of the KRAS mutation colorectal cancer cells.

\textcircled{1} Construction of the LoVo Cell Line with Stable Knock-Down KRAS

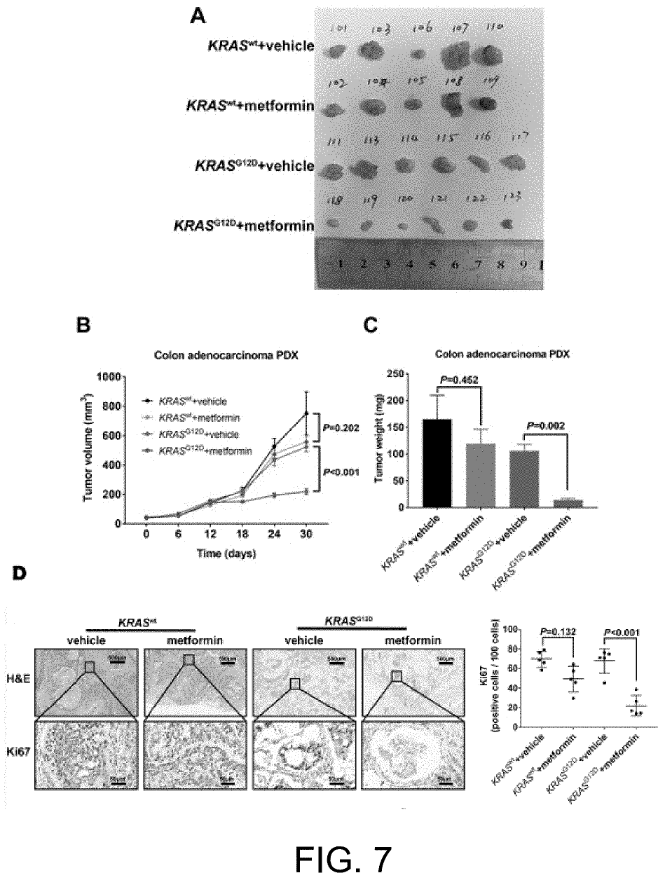

1) KRAS interference lentivirus (sh-KRAS) was constructed and packaged by Shanghai Genechem Co., LTD. 2 target sites were designed and shown in FIG. 7 specifically.

TABLE 7
Sequence information of the sh-KRAS target sites:

| | | Titer |
|---|---|---|
| NO. | Target Seq | (TU/mL) |
| KRAS- | ATGGTCCTAGTAGGAAATAAA | 1E+9 |
| RNAi(58761-1) | | |
| (SEQ ID | | |
| NO: 13) | | |
| KRAS- | TTGATGATGCCTTCTATACAT | 1E+9 |
| RNAi(58762-11) | | |
| (SEQ ID | | |
| NO: 14) | | |

2) LoVo cells were subcultured to a 6-well plate (about 1×10⁵/well), when cells were subjected to adherence growth, and the degree of cell fusion was not greater than 50%, fresh culture solution was replaced ahead of 2 h, and 10 μg/mL lentivirus infection enhancer polybrene was added. Multiplicity of Infection (MOI) of LoVo was about 20; lentivirus was added according to the formula: adding amount of lentivirus per well (μl)=MOI×cell population/titer (TU/ml)×1000. The culture solution was replaced after 16-18 h, and 1 μM puromycin was conventionally added for screening the cell line whose RNA stable expression was interfered by KRAS.

### 2. Experimental Results

Immunohistochemical results show that there are 18 cases of KRAS wildtype mCRC and 10 cases of KRAS mutation mCRC. As shown in FIG. 3C, the ratio of Ki67 (+) cells in the KRAS mutation mCRC tissues is obviously lower than that of the KRAS wildtype, thereby having a significant statistic difference (P<0.01).

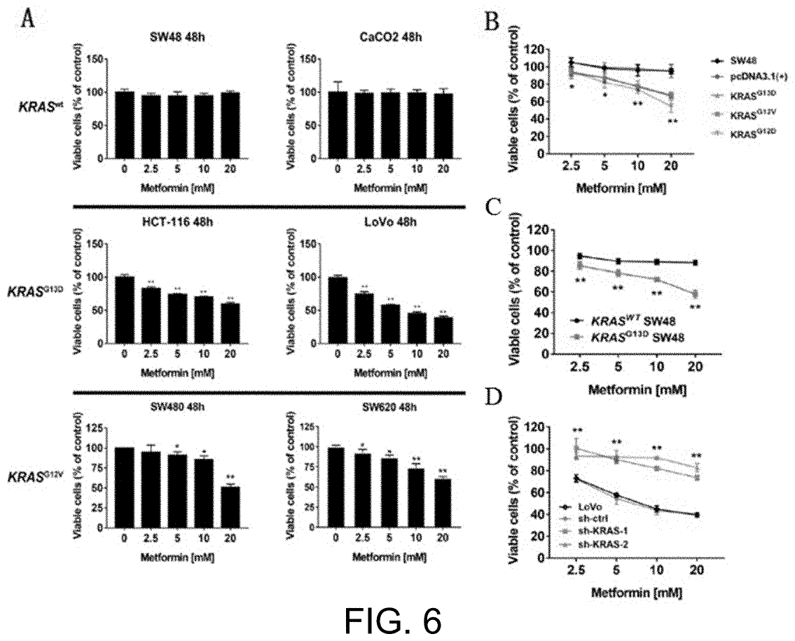

Cell experiment results show that metformin has an inhibiting effect on the cell viability of KRAS G13D, G12D and G12V mutation colorectal cancer cells, but has no effect (FIG. 6) on the KRAS wildtype colorectal cancer cells.

To sum up, the results show that compared with the KRAS wildtype, the KRAS mutation colorectal cancer patients have an extended overall survival and progression free survival after using metformin; further, the same results are verified by tumor tissue sections and cell viability experiments.

### Example 2 Verification of the Therapeutical Effect of Metformin on KRAS Mutation Tumor on a Tumor Animal Model

I. Construction of a PDX Model

Use of the PDX model: tumor tissues from the clinical KRAS wildtype and mutation CRC patients were taken, the tumor cells were cultured with a digestion process; after identifying the KRAS mutation, the tumor cells were planted in an axillary fossa area of nude mice.

### 1. Experimental Method

1) Collection of patients' tumor specimens: cancer tissues with non-necrotic edges were taken from specimens obtained after excision, and soaked into a PRMI1640 culture solution containing 5% fetal calf serum, 1×penicillin and streptomycin double-antibodies, and then transported at 4° C.;

2) the tumor tissues were cut into small pieces having a size of about 2×2×2 mm on a super clean bench, and cleaned for 3 times with the above culture solution to remove extravasated blood;

3) 4-6 week-age BALB/C nude mice were anesthetized with 4.8% chloral hydrate, and then an about 3 mm incision was cut on the underarm skin and subjected to blunt dissection with tweezers; and the trimmed tumor tissues were embedded subcutaneously, and the incision was sutured with 6-0 suture line, and a double-antibody was used to prevent wound infection;

4) 50 mg tumor tissues were taken to extract genome DNA for the identification of a KRAS genotype; and the remaining tissues were preserved at −80° C. for further use;

5) about 4-6 weeks later, about 1 cm³ nodulus was bulged on the location transplanted with the tumor, and the tumor tissue was taken out and trimmed into 2×2×2 mm tissue blocks for subculture. The subculture operation was the same as the above:

6) after being subcultured for 2-3 passages, nude mice were divided into 4 groups: KRAS wildtype tumor control group, KRAS wildtype tumor metformin group, KRAS mutation tumor control group, and KRAS mutation tumor metformin group, 10 pieces for each group;

7) when the transplanted tumor grew to 100-200 mm³, the nude mice were administered intragastrically everyday at 9:00 a.m. for consecutive 28 d. The control groups were administered normal saline intragastrically; the metformin groups were administered metformin solution intragastrically (based on the body weight of 100 mg/kg nude mice everyday, and dissolved by normal saline). The size of the transplanted tumor was measured everyday, volume (mm³)=[length×width²]/2;

8) the nude mice were killed 30 d layer, and tumor tissues were taken, shoot, measured, embedded and sliced.

### II. Therapeutical Effect of Metformin on KRAS Mutation Tumor

### 1. Experimental Method

200 mg/kg (equivalent to human 1000 mg) metformin was dissolved into water, and respectively taken by KRAS wildtype and mutation animals; then the tumor size was measured; 30 d later, the tumor tissues were killed and weighed.

### 2. Experimental Results

Compared with the KRAS wildtype tumor model, metformin may obviously inhibit the size and weight of the KRAS mutation tumor. Metformin has a better therapeutical effect on KRAS mutation colorectal cancer, which prompts that metformin may be selectively used by KRAS mutation patients clinically, thus providing a basis for the development of a medicament for the treatment of KRAS mutation patients (FIG. 7).

### Example 3 Inhibiting Effect of Metformin on the Proliferation of KRAS Mutation CRC Cells

I. Effect of Metformin on the Apoptosis of Colorectal Cancer Cells

Annexin V/PI double staining was used on the KRAS wildtype cell SW48 and KRAS (G13D) mutation cells LoVo to detect the effect of metformin on the apoptosis of colorectal cancer cells.

### 1. Experimental Method

1) The cells were inoculated on a 6-well plate and subjected to adherence growth for 12 h, starvation treatment over the night, and treated with the addition of an agent for 24 h, then the cells were digested by EDTA-free trypsin and washed for once with PBS without immobilization.

2) The cells were resuspended with the addition of 300 μl Binding Buffer, then 3 μl Annexin V-FITC and 3 μl Propidium Iodide (PI) were added and mixed well for incubation for 30 min in the dark at room temperature, then the cells were determined by a flow cytometry.

3) Flow cytometry detection of antibodies: apoptosis kit (A211-02) was purchased from KeyGEN; the detection kit (558662) for cell cycle PI simple staining was purchased from BD company.

### 2. Experimental Results

Annexin V/PI double staining was used to detect cell apoptosis. The result shows that 2.5 mM, 5 mM and 10 mM metformin do not accelerate the apoptosis of the KRAS wildtype CRC cells SW48 and KRAS mutation CRC cells LoVo.

II. Effect of Metformin on the Proliferation of CRC Cells

Edu was used to detect the ratio of proliferative cells, a plate clone formation experiment and PI/Rnase simple staining were used to detect the distribution of cell cycles, thus making the effect of metformin on the proliferation of CRC cells clear.

### 1. Experimental Method

(1) Proliferation Detection (Edu Method)

1) Cell treatment by Edu: cells were cultured in a culture dish (covered with a sterilized cover glass), and subjected adherence growth for 12 h, and starvation treatment over the night, and treated with the addition of an agent for the corresponding time, and then added with EdU (final concentration: 10 μM) at the last 2-6 h (EdU treatment time was determined according to the cell growth rate).

2) Immobilization: 4% paraformaldehyde was added according to 1 mL/well to immobilize the cells for 15 min at room temperature.

3) Slide washing: 3% BSA (dissolved by PBS) was used according to 1 mL/well for washing for twice.

4) Rupture of membrane: the cells were treated by 0.5% TritonX-100 according to 1 mL/well for 20 min at room temperature.

5) Slide washing: the same as the step 3).

6) Addition of a ClickiT reaction mixture: the reaction mixture was added per 504/piece in the dark for 30 min at room temperature.

7) Slide washing: 3% BSA (dissolved by PBS) was used according to 1 mL/well for washing for twice, then PBS was used for washing once again.

8) Cell nucleus staining: the cell nucleus was stained for 10 min in the dark with 50 μL DAPI (1:3000, PBS configuration).

9) Slide washing: PBS was used for washing twice.

10) The slide was dried in the dark and added with a quenching agent and sealed by nail polish.

11) The slide was observed under a full-automatic positive fluorescence microscope to analyze the result, and shoot and recorded (100×, 200×, 400×, the original image was kept).

(2) Plate Clone Formation

1) Cell inoculation: the culture solution was absorbed and discarded to collect the cells, then the cells were suspended in a treatment condition culture solution, and counted for 3 times to obtain a mean value, then the concentration of a cell suspension was adjusted to 1×10³/ml. 2.5 ml treatment condition culture solution was respectively added to per well of a 6-well culture plate; 0.5 ml cell suspension (namely, 500 cells per well) was added per well with a final volume of 3 ml. Note that the culture plate was shaken repeatedly according to a cross-shaped direction during inoculation, such that the cells were distributed evenly as much as possible.

2) Cell culture: after the cells were cultured for 2-3 week under standard conditions, then clone formation was observed, and the culture medium was replaced about every 3 d.

3) Clone staining: the culture was stopped when the cells formed visible clones (the number of cloned cells per well was about 50-150) by the naked eyes. The culture medium was discarded, and the clones were soaked and washed for twice with PBS carefully, 1.5 ml 4% paraformaldehyde was respectively added per well for immobilization for 15 min; then the immobilization solution was discarded, and the clones were cleaned with running water slowly; 1.5 ml crystal violet working solution was added per well, standing at room temperature, and the clones were stained for 30 min, and the staining solution was cleaned with running water slowly, then the remaining clones were dried in a draught cupboard.

4) Clone counting: the culture plate was put to a gel-imaging system to calculate the number of clones with the random software under a visible light; then images were scanned and preserved. Clone formation rate (%)=number of clones/number of inoculated cells×100%. Each set of cell sample was inoculated onto 3 repeated wells to perform the experiment for 3 times repeatedly and independently.

(3) Detection of Cell Cycle

1) The cells were inoculated onto a 6-well plate, and subjected to adherence growth for 12 h, then starvation treatment over the night, and treated with the addition of an agent for 24 h, then the cells were digested and washed for 3 times with PBS and centrifuged to discard supernatant; −20° C. pre-cooled 70% ethanol was added dropwise, and the cells were mixed well by vortex, and immobilized over the night at −20° C.

2) The cells were horizontally centrifuged for 10 min at 2000 rpm to discard the supernatant; then the cells were washed with PBS for 2-3 times to remove the residual ethanol with centrifugation for 5 min each time.

3) The cells were resuspended by 500 μL PI/RNase dyeing liquor, and incubated for 15 min in the dark, and put on a flow cytometry within 2 h for detection, and 488 nm wavelength was stimulated.

4) FlowJo 7.6 software was used for graphing analysis.

### 2. Experimental Results

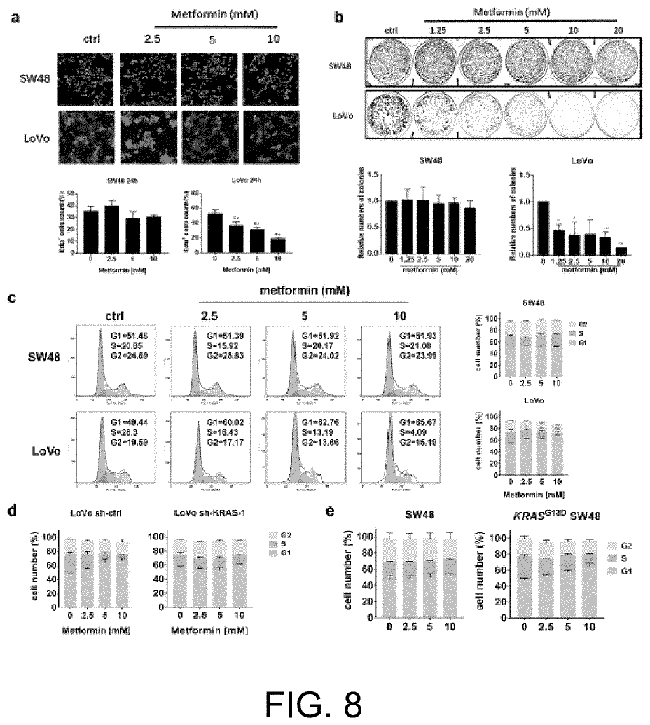

Metformin decreases the ratio of proliferating-phase cells (EdU positive) in the KRAS mutation CRC cells LoVo, inhibits the clone formation of LoVo clones, while has no obvious effect on the KRAS wildtype CRC cells SW48 (FIGS. 8A-B).

Flow cytometry result of the cell cycle shows that the concentration of metformin dependently increases the ratio of G1 cells of the KRAS mutation CRC cells LoVo, reduces the ratio of phase S cells, but has no obvious effect on the KRAS wildtype CRC cells SW48 (FIG. 8C).

Lentivirus shRNA was used to construct a LoVo cell line with knock-down KRAS protein. The result shows that the interference of KRAS (G13D) expression may down-regulate the inhibiting effect of metformin on LoVo cell viability, and down-regulate the inhibiting effect of metformin on the transformation of LoVo cells from G1 phase to phase S (FIG. 8D). The KRAS (G13D) mutation constructed via the CRISPR/Cas9 system enhances the inhibiting effect of metformin on the proliferation of tumor cells with cell cycle (FIG. 8E).

III. Mechanism of Metformin in Inhibiting the Proliferation of KRAS Mutation CRC Cells

Western blot was used to detect the change of metformin on the related molecules of sroliferative signaling pathways MEK/ERK/cyclin D1/RB and PI3K/AKT/mTOR/4E-BP1, which clarifies the mechanism of metformin in inhibiting the proliferation of KRAS mutation CRC cells.

### 1. Experimental Method

\textcircled{1} In Vitro Cell Experiment

1) Cells were inoculated on a culture dish and cultured to 60% of fusion degree, and treated with the addition of an agent for 24 h.

2) Protein was collected, and washed with PBS for 3 times, then added with 100 μl 1 RSDS buffer solution (100 mmol/1 Tris-Cl pH6.8, 2% SDS, 10% glycerinum), and at this time, the cell lysis solution was sticky.

3) The cell lysis solution was collected into a 0.5 ml centrifugal tube with a scraper, and cooked for 30 min at 100° C.

4) The total protein extractive in the cells was subjected to protein quantification according to a BCA kit process (BIO-RAD company).

5) The sample was treated according to a ratio of 9 μl denatured protein sample: 1 μl denatured buffer solution (β-mercaptoethanol (BME), 0.4% Bromphenol Blue), and boiled for 30 min at 100° C. for further use.

6) A prepared SDS-PAGE gel (10% separation gel and 5% stacking gel) was taken; samples were added with 40 ug/well, and subjected gel electrophoresis for 30 min at 80V, and gel electrophoresis for 90 min at 120V.

7) Membrane was transferred for 180 min at a constant current of 300 mA to transfer the protein on the gel onto the PVDF membrane.

8) The membrane was blocked for 90 min with 7% skimmed milk, and added with corresponding primary antibodies, and shaken at 4° C. over the night.

9) The membrane was washed for 3 times with TBST in the following day, 10 min each time, and then added with corresponding second antibodies for incubation for 4 h at 4° C.

10) The membrane was washed for 3 times with TBST, and then added with ECL for exposure.

\textcircled{2} Tumor tissues from PDX animals were taken and homogenated to extract protein for WB.

### 2. Experimental Results

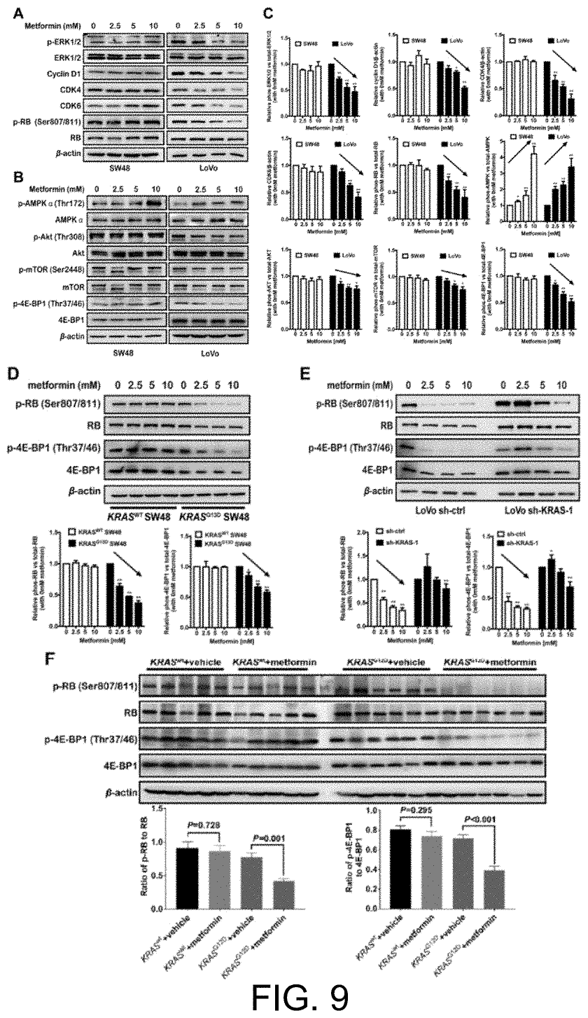

After being treated by metformin for 24 h, the KRAS mutation CRC cells LoVo have inhibited phosphorylation levels in ERK, RB, MPK, AKT, mTOR and 4E-BP1, but have no obvious change in the KRAS wildtype CRC cells SW48 (FIGS. 9A-C). The above results show that metformin may inhibit the ERK and AKT signaling pathways simultaneously, thus inhibiting the proliferation of KRAS mutation CRC cells.

For the KRAS (G13D) mutation SW48 cell line constructed via the CRISPR/Cas9 system, the change (FIG. 9D) of the cyclinD1/RB and AKT/mTOR/4E-BP1 signaling pathways is consistent with the KRAS mutation CRC cells LoVo. For the LoVo cell line with knock-down KRAS protein constructed via the lentivirus shRNA, the result shows that the interference of KRAS (G13D) expression may down-regulate the inhibiting effect of metformin on LoVo protein and 4E-BP1 phosphorylation (FIG. 9E).

Metformin inhibits the ERK and AKT signaling pathways of the KRAS mutation CRC cells in the PDX tumor tissues (FIG. 9F).

To sum up, metformin accelerates the stagnation of KRAS mutation CRC cells in phase G1, inhibits the proliferation of tumor cells instead of inducing cell apoptosis. Metformin may simultaneously inhibit ERK and AKT, and down-regulate phosphorylation of RB and 4E-BP1, thus inhibiting the KRAS mutation CRC cells.

The effect and mechanism of metformin on KRAS mutation CRC cells are confirmed for the first time. Meanwhile, the present invention also provides evidences for the combined inhibition of the two pathways of ERK/cyclin D1/RB and AKT/mTOR/4E-BP1 to enhance the therapeutic effect on the KRAS mutation CRC cells.

### Example 4 Sensitivity of Enhanced KRAS Mutation CRC Cells to Metformin

I. The Inhibiting Effect of Metformin on the Proliferation of CRC Cells is Correlated to the Intracellular Concentration Thereof

Lansoprazole was used to inhibit the cells to absorb metformin; and the cell viability was detected with CCK8 to confirm that the inhibiting effect of metformin on the proliferation of CRC cells was correlated to the intracellular concentration thereof.

The increase of concentration of metformin in KRAS mutation cells was verified by using the KRAS (G13D) mutation cells constructed via the CRISPR/Cas9 system and the LoVo cell line with stable knock-down KRAS.

### 1. Experimental Method

5 μM and 10 μM lansoprazole were used to treat HCT-116 and LoVo simultaneously; metformin was used to treat the cells at the same time for 48 h; the cell viability was detected by CCK8.

Metformin was used to detect the KRAS (G13D) mutation cells constructed via the CRISPR/Cas9 system and the LoVo cell line with stable knock-down KRAS to collect cell lysis solution at different time points, and the concentration of metformin in the cells was detected by mass spectrometry.

The concentration of metformin in KRAS wildtype and mutation tumor tissues was detected by mass spectrometry.

### 2. Experimental Results

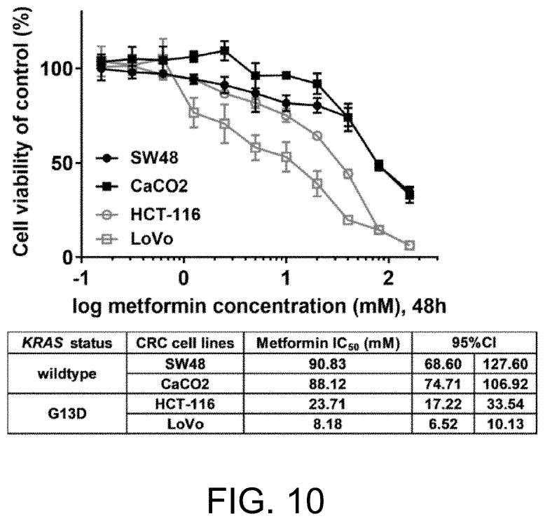

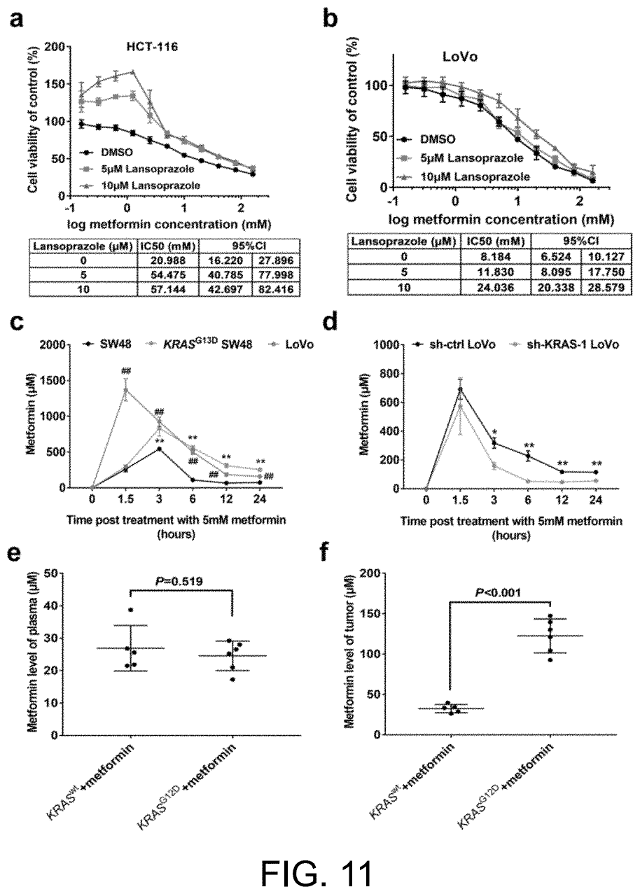

CCK8 results show that metformin has an IC50 of 90.83 mM (95% CI is 68.60-127.60), CaCO2 of 88.12 mM (95% CI is 74.71-106.92) to KRAS wildtype CRC cells SW48, while only has an IC50 of 23.71 mM (95% CI is 17.22-33.54), and LoVo of 8.18 mM (95% CI is 6.52-10.13) to the KRAS mutation CRC cells HCT-116 (FIG. 10). After lansoprazole was used to inhibit the cells to absorb metformin, IC50 of the HCT-116 and LoVo increases 1.2-2.5 times above (FIGS. 11A-B).

Mass spectrometry results show that compared with the control cell, the KRAS (G13D) mutation cells constructed via the CRISPR/Cas9 system have obviously increased concentration of metformin (FIG. 11C), while the concentration of metformin in the cells of the LoVo cell line with stable knock-down KRAS decreases obviously relative to LoVo (FIG. 11D).

Mass spectrometry results show that the concentration of metformin in the KRAS mutation tumor tissues is higher than that in the KRAS wildtype tumor tissues (FIG. 11F).

II. RNA Level of Metformin Channel Protein in Cells

RNA level of metformin channel protein in cells was detected by the analysis of the TCGA-COAD database to screen out the differential expression between the KRAS mutation CRC cell lines HCT-116 and LoVo, as well as between KRAS wildtype CRC cell lines SW48 and CaCO2.

### 1. Experimental Method

(1) Real-Time Quantitative PCR (RT-qPCR)

1) RNA Extraction

Specific steps were referring to the instruction of the RNA extraction kit (CW0581) of Beijing ComWin Biotech Co., Ltd.

2) Reverse Transcription

RNA concentration was measured by a Nanodrop ultraviolet spectrophotometer. 500 ng

NA was subjected to reverse transcription into cDNA, as follows:

Reaction system (10 μL) and reaction for 15 min at
37° C., and reaction for 5 s at 85° C.

| | | |
|---|---|---|
| 5× PrimeScript RT Enzyme Mix | 2 | μL |
| Total RNA | 500 | ng |

| | |
|---|---|
| ddH₂O | To 10 μL |

3) RT-qPCR

The instrument is a lightcycler fluorescent quantitative PCR or a CFX 96 fluorescent quantitative PCR from BIO-RAD. The reagent is a SYBR® Green I stain from Takara. Primers were designed by the primer-blast procedure from PubMed and synthesized by Life technology.

The primer sequences are shown in the table below:

| Primer name | Sequence (5′ to 3′) |
|---|---|
| homo-PMAT-F | AGTACCCAGGGACCTCCATC |
| (SEQ ID NO: 15) | |
| homo-PMAT-R | GTGTGCAGGGTCAGTCTCTC |
| (SEQ ID NO: 16) | |
| homo-OCT1-F | CCCCTCATTTTGTTTGCGGT |
| (SEQ ID NO: 17) | |
| homo-OCT1-R | TTTCTCCCAAGGTTCTCGGC |
| (SEQ ID NO: 18) | |
| homo-OCT2-F | AATCTCTACCCGCCTCCCTT |
| (SEQ ID NO: 19) | |
| homo-OCT2-R | CACAGAGCTCGTGAACCAGT |
| (SEQ ID NO: 20) | |
| homo-OCT3-F | CCCACTCCACCATCGTCAG |
| (SEQ ID NO: 21) | |
| homo-OCT3-R | ATCCTGCCATACCTGTCTGC |
| (SEQ ID NO: 22) | |
| homo-MATE1-F | GGAGTGATGGGTCTGTGGTC |
| (SEQ ID NO: 23) | |
| homo-MATE1-R | ACTCCGAGGCACGTTGTTTA |
| (SEQ ID NO: 24) | |
| homo-MATE2k-F | TGAGATCGGGAGCTTCCTCA |
| (SEQ ID NO: 25) | |
| homo-MATE2k-R | GAGCCCCAAGGGAATCATGT |
| (SEQ ID NO: 26) | |

The reaction procedure of the fluorescent quantitative PCR was as follows:

1) three-step approach (high efficiency but poor specificity):

\textcircled{1} predegeneration: the primers were treated at 95° C. for 30 s with 1 cycle;

\textcircled{2} PCR reaction: the primers were degenerated for 5 s at 95° C., and annealed for 30 s at 55° C., and extended for 45 s at 72° C. with 35 cycles;

\textcircled{1} dissociation curve analysis: the primers were treated respectively at 95° C. for 0 s, at 65° C. for 15 s, and at 95° C. for 0 s, with 1 cycle;

2) two-step approach (high specificity but low efficiency):

\textcircled{1} predegeneration: the primers were treated at 95° C. for 30 s with 1 cycle;

\textcircled{2} PCR reaction: the primers were degenerated for 5 s at 95° C., and annealed and extended for 30 s at 60° C., with 40 cycles;

\textcircled{3} a dissociation curve analysis: the primers were treated respectively at 95° C. for 0 s, at 65° C. for 20 s, and at 95° C. for 0 s, with 1 cycle;

(quoted from the instruction of the SYBR®Premix Ex Taq II reagent).

At the end of PCR, the dissociation curve was referenced to judge the specificity of the primers; based on the Cp value obtained by the reaction, calibration was performed according to standard curves and internal references of different genes; the control group was set 1 for analysis and graphing.

### 2. Experimental Results

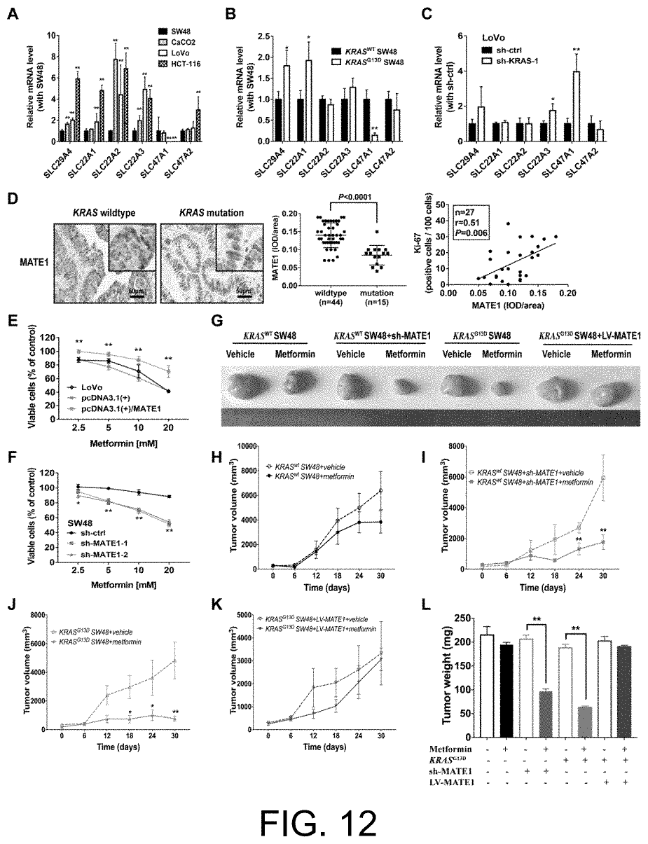

Based on the analysis of the TCGA-COAD database and screening of the RNA level of metformin passage in cell lines, it is found that compared with the KRAS wildtype CRC cells, the KRAS mutation CRC cell line (HCT-116 and LoVo) has decreased expression of MATE1 (SLC47A1) (FIG. 12A); The KRAS (G13D) mutation SW48 cell line has decreased expression of MATE1 (SLC47A1) (FIG. 12B); and the sh-KRAS-LoVo has increased expression of MATE1 (SLC47A1) (FIG. 12C).

III. Expression level of MATE1

The differentially expressed MATE1 was screened and verified on clinical specimens by immunohistochemistry via the over-expression or knock-down experiments of intracellular genes.

### 1. Experimental Method

The immunohistochemistry (labeled by p-RB) was the same as that in Example 1.

### 2. Experimental Results

Immunohistochemical results of the clinical specimens show that compared with the KRAS wildtype colorectal cancer, the KRAS mutation colorectal cancer has decreased MATE1 protein level; and in the clinical specimens of the mCRC patients after taking metformin, the expression of MATE1 is in direct proportion to the expression of the cell proliferation index p-RB (FIG. 12D).

MATE1 was over-expressed on LoVo for a forward experiment or MATE1 was interfered on SW48 for reverse experiment to prove the down-regulated expression of MATE1, thus accelerating the inhibiting effect of metformin on the proliferation of colorectal cancer cells (FIGS. 12E-F).

The expression of MATE1 and a transcription factor Sp1 was detected by Western blot. The results show that the expression of MATE1 in colorectal cancer cells is not correlated to the expression of Sp1.

IV. In Vivo Verification for the Tumor Inhibition Effect of Low Expression of MATE1 with Metformin

The tumor inhibition effect of MATE1 with metformin was verified by a cell-derived xenograft transplantation model (CDX) experiment.

### 1. Experimental Method

In the CDX, CRISPR/Cas9 was first utilized to build a KRAS^{G13D}SW48 cell line; MATE1 was knocked down by shRNA lentivirus transduction in SW48 to build a sh-MATE1-SW48 cell line; lentivirus was infected in KRAS^{G13D}SW48 cells to over-express MATE1. 1×10⁶ cells were suspended in a basement membrane matrix (100 μl basement membrane matrix and 100 μl PBS) and subcutaneously injected into BALB/c nude mice. CDX mice were randomly distributed into a metformin treatment group and a control group. 200 mg/kg (equivalent to human 1000 mg) metformin was dissolved into water, and respectively taken by the 4 groups of animals; then the tumor size was measured; 30 d later, the tumor tissues were killed and weighed.

### 2. Experimental Results

The result shows that in the SW48 CDX, there is no obvious anti-tumor effect in the metformin treatment group and in the control group; but when MATE1 was knocked down, the metformin significantly inhibits the tumor growth of SW48+sh-MATE1. On the contrary, compared with the KRAS^{G13D}SW48 tumor, metformin has no effect in the KRAS^{G13D}SW48 allogeneic graft after MATE1 was over-expressed (FIGS. 12G-L).

V. Difference in the Promoter CpG Island Methylation Level of the MATE1 Gene

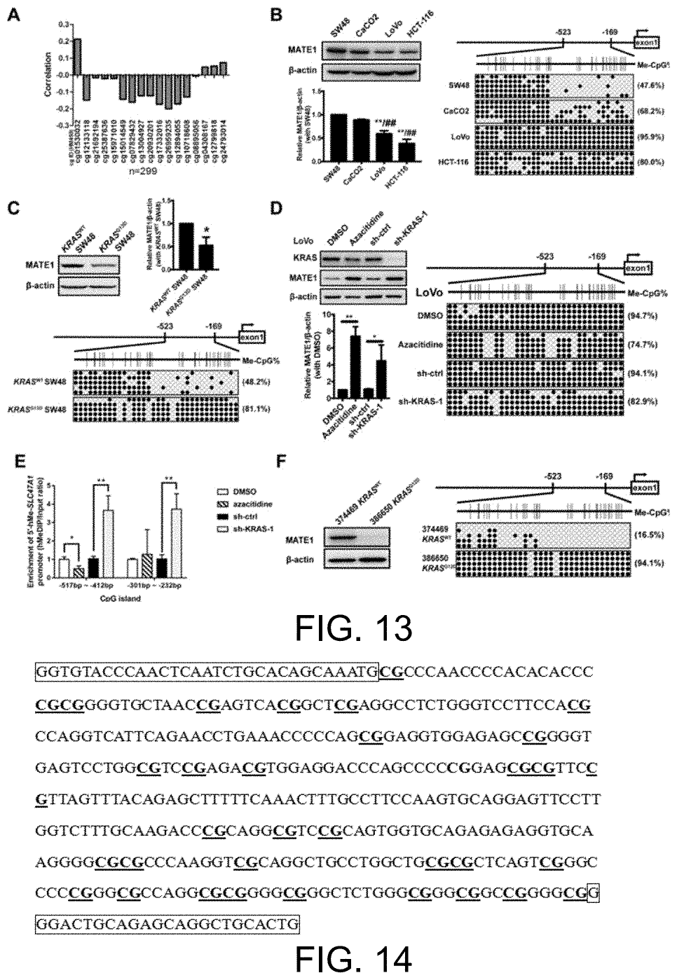

Analysis and relevant statistics were performed according to the mRNA-seq and MethyArray 450K data in the TCGA-COAD database to prompt that the expression of MATE1 in colorectal cancer cells is in negative correlation to the MATE1 promoter methylation (FIG. 13A).

A bisulfate sequencing method was used to detect the difference in the promoter CpG island methylation level of the MATE1 gene between the KRAS mutation CRC cell lines HCT-116 and LoVo, as well as between the KRAS wildtype CRC cell lines SW48 and CaCO2.

### 1. Experimental Method

(1) Bisulfite Sequencing PCR (BSP)

1) DNA was Modified with Sodium Bisulfite:

A. Genome DNA was extracted, and then subjected to 5mC>U sodium bisulfite reaction according to the following reaction system and conditions:

Reaction system (120 μL)

| | | |
|---|---|---|
| Bisulfite Mix | 90 | μL |
| Buffer solution DP | 10 | μL |
| DNA template | 1 | μg |

| | |
|---|---|
| ddH₂O | To 120 μL |

Reaction conditions

| Temperature | Time |
|---|---|
| 95° C. | 10 min |
| 64° C. | 60 min |
| 4° C. | ∞ |

B. DNA was purified after being treated by bisulfite by reference to the product instruction of the DNA bisulfite transformation kit (DP215).

2) PCR Amplification and Vector Binding:

A. After the genome DNA sample was modified by bisulfite, the MATE1 promoter CpG island was amplified. The sequence and amplification primers are shown in the table 14 below (the frame denotes primer sequences, and the underline denotes CpG sites).

PCR reaction system and conditions were as follows:

Reaction system (50 μL)

| | |
|---|---|
| 2× PrimeSTAR Max Premix | 25 μL |
| Forward primer | 1 μL |
| Reverse primer | 1 μL |
| DNA template is modified by bisulfite | 2 μL |
| ddH₂O | To 50 μL |

Reaction Conditions

| Process | Temperature | Time | Cycle |
|---|---|---|---|
| Denaturation | 98° C. | 10 s | 35cycle |
| Annealing | 55° C. | 5 s | |
| Extension | 72° C. | 45 s | |
| Preservation | 4° C. | ∞ | |

B. The cells were recovered and purified by a gel, then an A addition reaction was performed to the blunt ends PCR products, and linked into pGM-T of a T vector, and then transformed into competent cells for amplification; 5 monoclonal antibodies were picked out for sequencing; the specific steps were the same as that in Example 3.

3) Analysis on the methylation site and level: the sequencing results were directly analyzed with the website http://quma.cdb.riken.jp/QUantification tool for Methylation Analysis (QUMA); a dot diagram was selected. Each dot represents 1 CpG site, the white denotes non-methylation, the black denotes methylation, and there are 34 CpG sites in total.

(2) Hydroxymethylated DNA Quantification

1) Hydroxymethylated DNA Immunoprecipitation (hMeDIP);

A. Genome DNA was extracted and subjected to ultrasonication with 25% energy of Sonics Ultrasonic Cell Disruptor according to 10 s/pulsex4 pulses every other 40 s, and the genome DNA may be raptured into about 400 bp fragments.

B. The DNA fragments containing 5hmC were enriched by a hMeDIP ChIP kit from Abeam. The specific steps were referring to the instruction of the hMeDIP ChIP Kit (ab117134) from Abeam.

2) Semi-quantitative analysis by RT-qPCR: RT-qPCR primers are as follows, and the RT-qPCR reaction system and conditions were the same as the above. Non-enriched DNA was used as calibration.

| Primer name | Sequence (5′ to 3′) |
|---|---|
| CpG2-70bp-F | TTGCCTTCCAAGTGCAGGAGT |
| (SEQ ID NO: 27) | |
| CpG2-70bp-R | CTCTCTCTGCACCACTGCGG |
| (SEQ ID NO: 28) | |
| CpG1-106bp-F | CCCAACTCAATCTGCACAGC |
| (SEQ ID NO: 29) | |
| CpG1-106bp-R | CTGAATGACCTGGCGTGGAA |
| (SEQ ID NO: 30) | |

### 2. Experimental Results

By bisulfate sequencing PCR, it can be seen that compared with the KRAS wildtype CRC cells SW48 and CaCO2, the MATE1 promoter in the KRAS mutation CRC cells HCT-116 and LoVo has a higher methylation level (FIG. 13B).

VI. MATE1 Expression is Regulated by Controlling the Methylation Level of the MATE1 Promoter Via KRAS Mutation

LoVo cell line with knock-down KRAS constructed via lentivirus shRNA and the CRISPR/Cas9 system was used to construct a KRAS (G13D) mutation SW48 cell line were used to forward and reversely verify that the MATE1 expression is regulated by controlling the methylation level of the MATE1 promoter via KRAS mutation. The methylation level of the MATE1 in the PDX KRAS mutation and wildtype tumor tissues was detected.

### 1. Experimental Method

The methylation level was detected the same as the above.

### 2. Experimental Results

For the KRAS (G13D) mutation SW48 cell line constructed via the CRISPR/Cas9 system, the MATE1 promoter has an increased methylation level (FIG. 13C); for the LoVo cell line with knock-down KRAS constructed via the lentivirus shRNA, the MATE1 promoter has a decreased methylation level (FIGS. 13D-E); and the methylation level of MATE1 in the PDX KRAS mutation tumor tissues is obviously higher than that of the wildtype (FIG. 13F).

VII. Differential Expression and Verification of Transmethylases and Demethylases

The RNA level of the cell lines was detected to screen the differentially expressed transmethylases and demethylases; and the differentially expressed transmethylases and demethylases were verified in clinical specimens, the LoVo cell line with knock-down KRAS constructed via lentivirus shRNA, and the KRAS (G13D) mutation SW48 cell line constructed via the CRISPR/Cas9 system.

### 1. Experimental Method

RNA detection method is the same as the above; and the primers are as follows:

| Primer name | Sequence (5′ to 3′) |
|---|---|
| homo-DNMT1-F | GAGCTACCACGCAGACATCA |
| (SEQ ID NO: 31) | |
| homo-DNMT1-R | CGAGGAAGTAGAAGCGGTTG |
| (SEQ ID NO: 32) | |
| homo-DNMT3A-F | CAAGCGGGACGAGTGGCTGG |
| (SEQ ID NO: 33) | |
| homo-DNMT3A-R | TCAGTGGGCTGCTGCACAGC |
| (SEQ ID NO: 34) | |
| homo-DNMT3B-F | CTCAGAGGCAGTGACAGCAG |
| (SEQ ID NO: 35) | |
| homo-DNMT3B-R | TGTCTGAATTCCCGTTCTCC |
| (SEQ ID NO: 36) | |
| homo-TET1-F | ACCCCCTGTCACCTGCTGAGG |
| (SEQ ID NO: 37) | |
| homo-TET1-R | GCGATGGCCACCCCACCAAT |
| (SEQ ID NO: 38) | |
| homo-TET2-F | TCACACCAGGTGCACTTCTC |
| (SEQ ID NO: 39) | |
| homo-TET2-R | GGATGGTTGTGTTTGTGCTG |
| (SEQ ID NO: 40) | |
| homo-TET3-F | TCTCCCCAGTCTTACCTCCG |
| (SEQ ID NO: 41) | |
| homo-TET3-R | CCAGGCTTCAGGGAACTCAG |
| (SEQ ID NO: 42) | |

### 2. Experimental Results

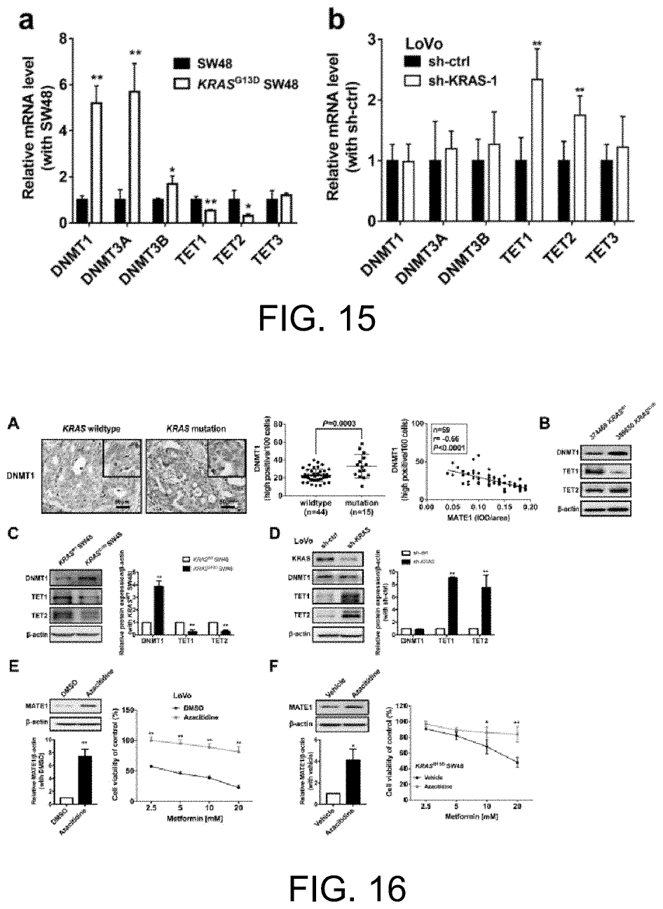

The differential expression of the transmethylases and demethylases in the cell lines was detected. The result prompts that the transmethylases DNMT1 and DNMT3A are up-regulated in KRAS mutation CRC cells, while the demethylase TET1/2 is down-regulated in the KRAS mutation CRC cells (FIG. 15A). TET1/2 is up-regulated in the LoVo cell line with knock-down KRAS constructed via lentivirus shRNA (FIG. 15B).

Meanwhile, histochemical stain was used to verify the expression of the DNMT1 and TET1 in 59 cases of colon cancer samples, and ImageJ software was used to calculate the ratio of the cells with strongly positive nuclear staining. The result shows that the protein level of DNMT1 in the KRAS mutation CRC tissues is higher than that of the KRAS wildtype (P=0.0003), while there is no significant difference in the expression of TET1 between the two groups (P=0.2989). The result of the Spearman rank correlation analysis shows that the ratio of the strongly positive DNMT1 cells is in significantly negative correlation to the expression of MATE1 (r=−0.66 P<0.0001, n=59) (FIG. 16A). The expression of MATE1 in KRAS mutation PDX tumor tissues increases, but the expression of TET1 decreases (FIG. 16B).

The results show that the DNMT1 expression increases, the TET1/2 expression decreases (FIG. 16C) in the KRAS (G13D) mutation SW48 cell line constructed via the CRISPR/Cas9 system; while in the LoVo cell line with knock-down KRAS constructed via lentivirus shRNA, the DNMT1 expression decreases and TET1/2 expression increases (FIG. 16D).

A methylase inhibitor azacitidine was used in the LoVo cells and KRAS (G13D) mutation SW48 cell line to up-regulate the expression of MATE1 in the KRAS mutation CRC cells, thus inhibiting the anti-tumor proliferation effect of metformin (FIGS. 16E-F). TET1/2 was interfered in the LoVo cell line with knock-down KRAS to down-regulate the expression of MATE1 once again, thus accelerating the inhibiting effect of metformin on the proliferation of tumor cells (FIG. 16G).

To sum up, MATE1 is down-regulated by KRAS mutation to improve the concentration of metformin in cells, thus enhancing the inhibiting effect of metformin on the proliferation of colorectal cancer cells. Metformin may be selectively used to treat colorectal cancer by detecting the genotype of KRAS No. 2 exon clinically.

In the end, it should be specified that the above examples are merely illustrative of the technical solution of the present invention, but not construed as limiting the protection scope of the present invention. A person skilled in the art may further make different forms of changes or alterations on the basis of the above description and thinking. All the embodiments need not and may not be described exhaustively here. Any modification, equivalent replacement and improvement and the like made within the spirit and principle of the present invention shall fall within the protection scope of the claims of the present invention.

What is claimed is:

1. Use of a MATE1 protein and/or an inhibitor of an encoding gene SLC47A1 thereof in the synergistic treatment of colorectal cancer with metformin or in the preparation of a medicament for the synergistic treatment of colorectal cancer with metformin.

2. The use according to claim 1, wherein the MATE1 protein and/or the inhibitor of an encoding gene SLC47A1 thereof comprises: one or a mixture of several of a transmethylase DNMT1 or an encoding gene thereof, a transmethylase DNMT3A or an encoding gene thereof, an activating agent of the transmethylase DNMT1 or the encoding gene thereof, an activating agent of the transmethylase DNMT3A or the encoding gene thereof, an inhibitor of a demethylase TET1, an inhibitor of a demethylase TET2, an inhibitor of the encoding gene of the demethylase TET1, and/or an inhibitor of the encoding gene of the demethylase TET2.

3. A pharmaceutical composition for treating colorectal cancer, wherein the pharmaceutical composition contains metformin, and a MATE1 protein and/or an inhibitor of an encoding gene SLC47A1 thereof.

4. A method for treating colorectal cancer, wherein metformin is used in combination with a MATE1 protein and/or an inhibitor of an encoding gene SLC47A1 thereof.

5. Use of a MATE1 protein and/or an inhibitor of an encoding gene SLC47A1 thereof as a synergist for the treatment of colorectal cancer with metformin.
